(19) United States
(12) Patent Application Publication
(10) Pub. No.: US 2026/0263144 A1
(43) Pub. Date: Sep. 10, 2026

(54) ELECTROSURGICAL INSTRUMENT AND ELECTROSURGICAL APPARATUS

(71) Applicant: Creo Medical Limited, Chepstow (GB)

(72) Inventors: Christopher Paul HANCOCK, Chepstow (GB); Louis TURNER, Chepstow (GB); David WEBB, Chepstow (GB); Steven THOMAS, Chepstow (GB); George Christian ULLRICH, Chepstow (GB); Warren JONES, Chepstow (GB); Philip ANTHONY, Chepstow (GB); Duncan James Foster FITZSIMONS, St. Albans (GB)

(21) Appl. No.: 19/161,155

(22) PCT Filed: Feb. 29, 2024
(86) PCT No.: PCT/EP2024/055265

§ 371 Date: Aug. 29, 2025

(30) Foreign Application Priority Data
Mar. 3, 2023 (GB) 2303160.2
Mar. 3, 2023 (GB) 2303162.8
Jun. 19, 2023 (GB) 2309167.1

(51) Int. Cl. A61B 18/14; A61B 17/00; A61B 18/00; A61B 18/18

(52) U.S. Cl. CPC A61B 18/1445; A61B 2017/00862; A61B 2018/00083; A61B 2018/0063; A61B 2018/00994; A61B 2018/1455; A61B 2018/1467; A61B 2018/1861

(57) ABSTRACT

An electrosurgical instrument for sealing and/or transecting tissue comprises an elongated shaft that houses a transmission line for conveying microwave and/or radio-frequency energy to a pair of opposing jaws. A first jaw supports first and second tissue-contacting electrodes, which are electrically separated by an elastically deformable isolating portion that allows the first electrode to shift relative to the second electrode under clamping pressure. A second jaw carries a third electrode positioned to oppose the first and second electrodes. The jaws are movable between an open position, enabling tissue placement between their respective tissue-engaging surfaces, and a closed position, in which the surfaces clamp the tissue. Selective energization of the electrodes with microwave and/or radio-frequency energy seals, coagulates, and/or cuts the compressed tissue. The elastic isolator compensates for variations in tissue thickness and applied clamp force while preserving the desired electrical spacing between the first and second electrodes.

### FIELD OF THE INVENTION

The invention relates to an electrosurgical instrument for sealing and/or cutting tissue. The electrosurgical instrument can be configured to grasp biological tissue and deliver microwave energy into the grasped tissue to seal the tissue by coagulation or cauterisation. The electrosurgical instrument may be used to apply pressure to close one or more blood vessels before applying electromagnetic radiation (preferably microwave energy) to seal the blood vessel(s). The electrosurgical instrument may also be arranged to cut, e.g. separate or divide, the vessel or surrounding tissue after coagulation or sealing, e.g. using radiofrequency (RF) energy, microwave energy, and/or a mechanical cutting element, such as a blade. The invention may be applied to a vessel sealer for use in laparoscopic surgery or open surgery as well as to an endoscopic instrument.

The invention also relates to an electrosurgical apparatus for sealing and/or cutting tissue which comprises a generator unit for generating radiofrequency and microwave electromagnetic energy, and the electrosurgical instrument.

### BACKGROUND TO THE INVENTION

Electrosurgical instruments for delivering heat energy into grasped biological tissue are known. For example, it is known to deliver microwave energy from a bipolar electrode arrangement in the jaws of a forceps. The microwave energy may be used to seal a vessel by thermal denaturation of extracellular matrix proteins (e.g. collagen) within the vessel wall. The heat energy may also cauterise the grasped tissue and facilitate coagulation.

Such devices typically find application on the end of minimally invasive surgical laparoscopic tools but can equally find use in other clinical procedural areas such as gynaecology, endourology, gastrointestinal surgery, ENT procedures, or endoscopic procedures. Depending on the context of use, these devices can have differing physical construction, size, scale and complexity.

For example, a gastrointestinal instrument might be nominally of 3 mm diameter mounted on to the end of a very long flexible shaft. In contrast, a laparoscopic instrument may be used on the end of an industry standard nominal 5 mm or 10 mm diameter rigid or steerable steel shaft.

U.S. Pat. No. 6,585,735 describes an endoscopic bipolar forceps in which the jaws of the forceps are arranged to conduct bipolar energy through the tissue held therebetween.

EP 2 233 098 describes microwave forceps for sealing tissue in which the sealing surfaces of the jaws include one or more microwave antennas for radiating microwave energy into tissue grasped between the jaws of the forceps.

WO 2015/097472 describes electrosurgical forceps in which one or more pairs of non-resonant unbalanced lossy transmission line structures are arranged on the inner surface of a pair of jaws.

### SUMMARY OF THE INVENTION

At its most general, the present invention provides various types of electrosurgical instruments that can enable fine tissue cutting and dissection to be performed on tissue.

Moreover, the electrosurgical instruments may provide additional functionality, such as sealing biological tissue, such as (blood) vessels, using a confined microwave field that can yield a well-defined seal location with low thermal margin. With these additional functions, fewer device interchanges may be needed during a procedure.

The electrosurgical instruments disclosed herein may be used in any type of surgical procedure, but it is expected to find particular utility for non-invasive or minimally invasive procedures. For example, the device may be configured to be introduced to a treatment site through an instrument channel of a surgical scoping device, such as a laparoscope or an endoscope.

According to a first aspect of the present invention, there is provided an electrosurgical instrument for sealing and/or cutting tissue which comprises an instrument shaft, a first jaw, a second jaw, a first electrode, a second electrode, a third electrode, and a first isolating portion. The instrument shaft comprises a (coaxial) transmission line for conveying microwave electromagnetic energy and/or radiofrequency electromagnetic energy. The first jaw is attached to the instrument shaft and includes a first surface. The second jaw is attached to the instrument shaft and includes a second surface. The first, second, and/or third electrodes are configured to emit microwave and/or radiofrequency electromagnetic energy. The first isolating portion electrically isolates the first electrode from the second electrode. The first jaw and the second jaw can be moved between an open position, in which the tissue can be inserted between the first surface and the second surface, and a closed position, in which the first and second surfaces are brought together to clamp, hold, and/or grasp tissue therebetween. The first electrode and the second electrode are arranged on the first jaw. The first electrode is optionally exposed on the first isolating portion or can be completely buried within the first isolating portion. The third electrode is arranged on the second jaw. The first isolating portion is made from an elastically deformable material. The first electrode is attached to and supported by the first isolating portion so that the first electrode is movable relative to the second electrode upon application of pressure on the first electrode. Optionally, the second electrode and the third electrode are arranged on opposing sides of the first electrode in the closed position and are electrically connected to each other.

The first electrode may have a dual functionality of the emitting microwave energy and radiofrequency energy. The second and/or the third electrode may act as a return electrode when the first electrode is an active electrode for radiofrequency cutting. Further, the first electrode and the second electrode can seal tissue by the emission of microwave electromagnetic energy.

In use, the electrosurgical instrument may thus perform vessel/tissue sealing and/or vessel/tissue dividing. Vessel/tissue sealing is typically the application of pressure to squash the walls of a biological vessel together, followed by the application of some form of thermal energy. In the invention, the thermal energy is applied by the first, second, and/or third electrodes to the gripped tissue using the microwave electromagnetic energy. The pressure to the tissue can be applied by the first, second, and/or third electrodes (e.g. the first surface and second surface) and/or other parts of the first and second jaws. The applied electromagnetic energy disrupts/denatures the tissue cells and forms an amalgam of collagen predominant in vessel/tissue walls, which effectively bonds the vessel/tissue walls together. With time, post operatively, cellular recovery and regrowth occurs to reinforce the seal further.

Vessel/tissue dividing is a process of cutting through a continuous biological vessel/tissue to separate it into two pieces. It is normally performed after a vessel/tissue is first sealed. Vessel/tissue dividing can be performed by the first electrode as an active electrode. The vessel/tissue dividing can occur at the same position as vessel/tissue sealing.

The first electrode can be considered floating in the first isolating portion. In other words, the first electrode can be solely attached to the first jaw via the first isolating portion. For example, there may be no structural parts which support the first electrode on first jaw other than the first isolating portion.

A flexible electrical connection (e.g. a flexible wire) may provide the electrical connection between the first electrode and the transmission line. So, the first electrode is movable relative to the second electrode or other components of the first jaw. The movement of the first electrode is associated by a compression or stretch of the elastically deformable first isolating portion. For example, if a pressure is applied to the first electrode so that the first electrode is pressed into the first isolating portion (e.g. when tissue is grasped between the first jaw and the second jaw), the first isolating portion is compressed and the first electrode is moved away from the second jaw or relative to the second electrode (which can be fixed on the first jaw).

This can reduce the pressure on the tissue when sealing the tissue using microwave radiation. This is for example relevant if the first electrode protrudes from the first surface of the first jaw so that the pressure between the first electrode and the second jaw is higher compared other regions of the first surface and the second jaw. Further, the compression of the first isolating portion provides a force further compressing the tissue between the first electrode and the second jaw which may be helpful for radiofrequency cutting. For example, by virtue of the elastically deformable nature of the first isolating portion, the first electrode may be movable towards the second electrode when pressure is applied on the first electrode. Additionally, by virtue of the resilient nature of the first isolating portion, the first isolating portion may apply a reaction or return force which moves (or biases) the first electrode away from the second electrode.

The term “elastic” describes a body or material which can resist a distorting influence and can return to its original size and shape when that influence or force is removed. Solid bodies will deform when adequate loads are applied to them; if the material is elastic, the object will return to its initial shape and size after removal of the load.

The second electrode and the third electrode may each provide half-shells for containing the microwave energy emitted by the first electrode therein. The first electrode may be sandwiched between or (completely) surrounded by the second electrode and the third electrode in a closed position. The second jaw may include a second isolating portion which electrically isolates the first electrode from the third electrode in the closed position.

Optionally, the first electrode is exposed on the first isolating portion. The first electrode may include a ridge or bar made from an electrically conductive material. The first electrode can protrude from the first isolating portion. This means that the first electrode is not flush with the first isolating portion. The second jaw may come into contact with the first electrode but not with the first isolating portion. This results in that tissue clamped between the first jaw and the second jaw is compressed more at the first electrode compared to the first isolating portion. This may be helpful for locally increasing the pressure for radiofrequency cutting at the location of the radiofrequency cutting. However, this effect can be offset by the elastic compression of the first isolating portion as described above.

The first electrode can be flush with the first isolating portion. In this case, the area of the first surface around the first electrode may be elevated compared to the rest of the first surface. For example, the first isolating portion is angled in a cross-sectional view. In this embodiment, the elevated area of first isolating portion and the first electrode may be moved/compressed as described above.

Herein, the terms “proximal” and “distal” refer to the ends of the electrosurgical instrument, the shaft, and/or the coaxial transmission line further from and closer to a treatment site respectively. Thus, in use the proximal end is closer to a generator unit for providing the RF and/or microwave energy, whereas the distal end is closer to the treatment site, i.e. the patient.

The term “conductive” is used herein to mean electrically conductive, unless the context dictates otherwise.

The term “longitudinal” used below refers to the direction along the instrument channel parallel to the axis of the coaxial transmission line. The term “lateral” refers to a direction that is perpendicular to the longitudinal direction. The term “inner” means radially closer to the centre (e.g. axis) of the instrument channel. The term “outer” means radially further from the centre (axis) of the instrument channel.

The term “electrosurgical” is used in relation an instrument, apparatus or tool which is used during surgery and which utilises (bipolar) radiofrequency (RF) electromagnetic (EM) energy and/or microwave EM energy. Herein, RF EM energy may mean a stable fixed frequency in a range 10 kHz to 300 MHz, preferably in a range from 100 kHz to 5 MHz, and more preferably in a range from 360 to 440 kHz. The microwave EM energy may mean electromagnetic energy having a stable fixed frequency in the range 300 MHz to 100 GHz. The RF EM energy should have a frequency high enough to prevent the energy from causing nerve stimulation. In use, the magnitude of the RF EM energy and the duration for which it is applied may be selected to prevent the energy from causing tissue blanching or unnecessary thermal margin or damage to the tissue structure. Preferred spot frequencies for the RF EM energy include any one or more of: 100 kHz, 250 kHz, 400 kHz, 500 kHz, 1 MHz, 5 MHz. Preferred spot frequencies for the microwave EM energy include 915 MHz, 2.45 GHZ, 5.8 GHz, 14.5 GHZ, 24 GHz. 2.45 GHz and/or 5.8 GHz may be preferred.

The microwave electromagnetic energy and the radiofrequency electromagnetic energy may be conveyed along a common signal pathway through the instrument shaft. For example, a coaxial cable may provide the common signal pathway for conveying both the microwave energy and the radiofrequency energy. In this arrangement, the transmission line may comprise an inductive filter for blocking the microwave energy from the cutting element, and a capacitive filter for blocking the radiofrequency energy from the first and second electrodes. In an alternative arrangement, the radiofrequency energy and microwave energy are conveyed along separate pathways within the instrument shaft (the transmission line includes separate pathways), wherein the inductive filter and capacitive filter are provided at a proximal end of the instrument shaft, e.g. in a handle. For example, a coaxial cable is provided for conveying the microwave electromagnetic energy while two or more wires are provided for conveying the radiofrequency electromagnetic energy.

The instrument shaft may be dimensioned to fit within an instrument channel of a surgical scoping device. The surgical scoping device may be a laparoscope or an endoscope. Surgical scoping devices are typically provided with an insertion tube that is a rigid or flexible (e.g. steerable) conduit that is introduced into a patient's body during an invasive procedure. The insertion tube may include the instrument channel and an optical channel (e.g. for transmitting light to illuminate and/or capture images of a treatment site at the distal end of the insertion tube). The instrument channel may have a diameter suitable for receiving invasive surgical tools. The diameter of the instrument channel may be equal to or less than 13 mm, preferably equal to or less than 10 mm, and more preferably, especially for flexible insertion tubes, equal to or less than 5 mm.

The instrument shaft and the transmission line may be flexible so that they can be inserted into the instrument channel of the scoping device. Further, the transmission line may be arranged within a lumen of the shaft. The instrument shaft may cover and/or shield the transmission line. The transmission line may extend from a distal end to a proximal end of the electrosurgical instrument. In particular, the transmission line electrically connects the first electrode and the second electrode to the generator unit.

The electrosurgical instrument discussed herein may find applicability in other tissue welding techniques. For example, the energy delivery structure may be used as an alternative to staples. In some abdominal procedures, staple guns are used to deliver 50 to 100 small staples that are fired simultaneously between jaws that can have a length of 70 mm or more, or from an annular jawed arrangement with diameters of 20 to 50 mm. In this type of application multiple antenna structures such as those discussed herein may be used to cover the required length. The antenna structures may be arranged in any number of array forms to be activated simultaneously, sequentially or progressively in a suitable manner.

The first jaw and/or the second jaw may be movable relative to their instrument shaft. The first jaw and/or the second jaw may be attached to the instrument shaft via a joint or a hinge. The joint may include a pivot axis around which the first jaw and/or the second jaw may rotate. The first jaw and/or the second jaw may be activated by one or more actuation rods or control wires respectively connected to the first jaw and/or the second jaw. The one or more actuation rods or control wires may extend within the instrument shaft to a proximal end of the electrosurgical instrument. The one or more actuation rods may be connected to a handle with which the first and/or second jaws can be actuated, e.g. opened and/or closed. The electrosurgical instrument comprises an actuation mechanism which converts a back-and-forth movement of the actuation rod(s) or control wire(s) into a rotational movement of the first jaw and/or the second jaw.

For example, both jaws can be movable, e.g. rotatable around a (common) pivot axle. In another embodiment, one of the jaws is fixed to the shaft and the other jaw is movable relative to the one jaw.

In the open position, the first jaw and the second jaw are (maximally) spaced apart so that there is a free space between the first surface of the first jaw and the second surface of the second jaw. In this way, tissue can be inserted between the first surface and the second surface in the open position. Usually, the first jaw and the second jaw are moved towards the tissue such that the tissue is pushed into the space between the first surface and the second surface in the open position of the first jaw and the second jaw.

By moving the first jaw and/or the second jaw from the open position to the closed position, the tissue between the first surface and the second surface can be grasped and/or clamped between the first surface and the second surface. In this way, the tissue can be fixed between the first surface and the second surface in the closed position. The first surface and the second surface are the faces of the first jaw and the second jaw, respectively, that face each other in the open and/or closed position. The tissue contacts the first surface and the second surface in the closed position.

The pair of jaws may be pivotable relative to each other about the pivot axis that lies transverse to a longitudinal axis of the coaxial transmission line. In one example, the pair of jaws comprises a static jaw that is fixed relative to the instrument shaft, and a movable jaw that is pivotably mounted relative to the static jaw to open and close the gap between the opposing inner surfaces. The energy delivery structure may be disposed on the inner surface of the static jaw. In another example, both jaws are arranged to pivot with respect to the instrument shaft, e.g. in a symmetrical forceps-type or scissors-type arrangement. Relative movement of the pair of jaws may be controlled from a handle at a proximal end of the instrument shaft. A control rod or control wires may pass through the instrument shaft to operably couple an actuation mechanism on the handle to the pair of jaws.

In another example, the pair of jaws may be arranged to move relative to one another in a manner that maintains the inner surfaces thereof in an aligned, e.g. parallel, orientation. This configuration may be desirable for maintaining a uniform pressure on grasped tissue along the length of the jaws. One example of such a closure mechanism is disclosed in WO 2015/097472.

The first jaw and/or the second jaw may have a Maryland configuration. This can include that the first jaw and the second jaw are not straight but bent/curved, e.g. forming an arc or an S-shape in a side view.

In an optional embodiment, the first isolating portion is made from a material that is more elastic than a material from which the first electrode is made and/or from which the second electrode is made.

The first electrode and/or the second electrode can each be made from one or more materials which each are more rigid (e.g. have a higher rigidity) compared to the material (or materials) from which the first isolating portion is made. Optionally, a portion of the first isolating portion that is in contact with and/or surrounds the first electrode is more elastic than the materials from which to first electrode and/or the second electrode is made.

As a result, upon the application of a pressure on the first electrode, the first isolating portion or a portion of the first isolating portion elastically deforms whereas the first electrode and/or the second electrode do not or elastically deform to a much lower degree (e.g. by 1%, 5%, or 10% compared to the elastic deformation of the first isolating portion).

In an optional embodiment, the first isolating portion is made from silicone. Silicone or polysiloxane is a polymer made up of siloxane (—R2Si—O—SiR2-, where R can be organic group). The silicone and/or any other material suitable for the first isolating portion can be a rubber-like substance and can provide thermal insulation and/or electrical insulation. Further, the silicone used for the first isolating portion can provide non-stick characteristics which can be understood in that the silicone material used to form the first isolating portion reduces or provides less adhesion between the first isolating portion and the tissue in contact with the first isolating portion compared to other materials used for the first isolating portion.

The first isolating portion may be entirely or completely made from silicone.

In an optional embodiment, the second electrode and/or the third electrode each include a channel. Optionally, the first isolating portion is arranged in the channel and/or the first isolating portion interlocks with the second electrode.

The second electrode may form the outer surface of the first jaw and/or may provide the connection to the instrument shaft. Similarly, the third electrode may form the outer surface of the second jaw and/or may provide the connection to the instrument shaft. The channels of the second electrode and/or the third electrode may be provided by the shape of the second electrode and/or the third electrode, respectively. For example, the second electrode and/or the third electrode are half-shells. The channel may then correspond to the concave inner surface of the half-shell.

Alternatively, the second electrode and/or the third electrode may be a solid body having an elongate slot or recess which forms the channel. The second electrode and/or the third electrode may have a U-shape in a cross-sectional view perpendicular to the longitudinal direction. Side surface(s) of the second electrode may be exposed at the first face and/or side surface(s) of the third electrode may be exposed at the second face. For example, side surfaces of the second electrode are exposed at the first surface on each side of the first electrode.

The channel may extend along the longitudinal direction of the first jaw, the second jaw and/or the shaft. The channel can be open or close at the distal end of the second electrode, the third electrode, the first jaw, and/or the second jaw. The channel of the second electrode may be filled with the first isolating portion for electrically insulating the first electrode from the second electrode arranged in the channel of the second electrode. Further, a second isolating portion can be provided in the channel of the third electrode. Thus, the channel of the third electrode may be filled with the second isolating portion for electrically insulating the third electrode from a fifth electrode arranged in the channel of the third electrode.

In an optional embodiment, the electrosurgical instrument further comprises a second isolating portion which is arranged on the second jaw.

A surface of the second isolating portion that is exposed on the second jaw may form or is part of the second surface. The second isolating portion arranged on the second jaw may be made from the same electrically non-conductive material as the first isolating portion. The first isolating portion is attached to the second electrode, for example by adhesion and/or by form fit (positive fit, positive mechanical engagement, or positive interlocking). For example, one or more protrusions and/or one or more recesses are provided in the channel of the second electrode which provide an undercut for the first isolating portion. A form fit may be used for attaching the first isolating portion to the second electrode in case silicone is used for the first isolating portion because silicone is non-stick so that it is less suitable for being adhered to the second electrode. For example, the first isolating portion in a fluid state is poured into the channel of the second electrode and then cured or hardened so that it interlocks with the one or more protrusions and/or one or more recesses provided in the channel of the second electrode. The second isolating portion may be attached to the third electrode similar or identical to the way that the first isolating portion is attached to the second electrode (e.g. by form fit).

In an optional embodiment, the first electrode is (completely or entirely) made from an electrically conductive material and interlocks with the first isolating portion.

The first electrode may have a shape of a rod or bar. Generally, the first electrode may be an elongate body which extends along the direction of extension (longitudinal direction) of the first jaw and/or the second jaw. The first electrode may be (entirely) made from a metal material.

In an optional embodiment, the first electrode includes at least one through-hole which is filled with the first isolating portion.

The first electrode may include one or more through-holes which provide a form fit attachment of the first electrode to the first isolating portion. For example, the first electrode is held within the channel of the second electrode without contacting the second electrode. Then, the first isolating portion in a fluid state is poured into the channel of the second electrode and then cured or hardened so that the first isolating portion interlocks with the first electrode. In this way, the first isolating portion supports and fixes the first electrode to the second electrode while, at the same time, the first electrode is electrically isolated from the second electrode.

In an optional embodiment, the first electrode includes a base plate and a rib arranged on the base plate for forming a T-shape in a cross-sectional view of the first electrode. Optionally, at least a part of the base plate on each side of the rib and facing the second jaw is covered by the first isolating portion.

The base plate and/or the rib may each have an elongate shape and can be made from a plate or strip of electrically conductive material such as metal. The base plate can be (permanently) attached to the rib, for example by welding. Alternatively, the base plate and the rib are a unitary component.

A section or portion of the first electrode (e.g. of the rib) may be exposed at the first surface. The base plate may be provided for interlocking the first electrode with the first isolating portion. For example, the base plate protrudes on one or both sides from the rib. For example, a width of the base plate is larger than a thickness of the rib wherein the width and the thickness are measured in the same direction in an assembled state of the first electrode. The base plate may extend perpendicular to the rib. The base plate may be attached to the rib at an end of the rib which is opposite to the end of the rib that is exposed at the first surface. The base plate may be fully embedded in the first isolating portion.

The provision of the base plate may be a means for interlocking the first electrode with the first isolating portion and may be provided as an alternative or in addition to the one or more through-holes described above. The base plate may provide an undercut for the rib.

The base plate may include a side surface which faces the second jaw in the closed position. The other side surface of the baseplate may face away from the second jaw in the closed position and/or faces a bottom of the channel of the second electrode. A part of the side of the baseplate that faces the second jaw in the closed position is covered by the first isolating portion. This means, that a section of the first isolating portion is provided between the first surface and that side surface of the base plate. As such, the first electrode interlocks with the first isolating portion using the base plate as an undercut.

Optionally, the first isolating portion may include a cavity in the direction of extension of the first jaw which has T-shape in a cross-sectional view. The first electrode is arranged in the T-shaped cavity of the first isolating portion. As described above, the first isolating portion can be formed by pouring the first isolating portion (in a fluid state) in the channel of the second electrode while holding the first electrode in its desired position (e.g. relative to the second electrode).

In an optional embodiment, the electrosurgical instrument further comprises a fifth electrode for emitting microwave and/or radiofrequency energy and arranged on the second jaw. Optionally, the fifth electrode includes a base plate and a rib arranged on the base plate for forming a T-shape in a cross-sectional view of the fifth electrode. Further optionally, the first electrode is completely embedded within the first isolating portion and/or the fifth electrode is completely embedded within the second isolating portion.

In an exemplary embodiment, the first electrode and the fifth electrode may both be active electrodes for microwave sealing. For example, the first electrode and the fifth electrode may be electrically connected to each other or maybe electrically connected to the same conductor of the transmission line, e.g. an inner conductor of the coaxial cable of transmission line.

The T-shaped fifth electrode may have to same characteristics, optional features, and/or optional embodiments as the T-shaped first electrode. So the same comments and/or remarks made in connection with the first electrode can equally apply to fifth electrode. With the fifth electrode, the base plate provides an undercut to the rib so that the fifth electrode is supported by and attached to the second isolating portion.

Optionally, the first electrode and/or the fifth electrode may not be exposed on the first surface and/or the second surface, respectively. The first electrode and/or the fifth electrode may be completely immersed, buried, and/or covered within/by the first isolating portion and/or the second isolating portion, respectively. So, the first electrode and/or the fifth electrode may never be in contact with tissue grasped between the first jaw and the second jaw. This avoids the risk that the first electrode and/or the fifth electrode may stick to the tissue.

Further, the first electrode and/or the fifth electrode may not be buried deep in the first isolating portion and/or the second isolating portion, respectively. Rather, only a thin section of the first isolating portion and/or the second isolating portion covers the first electrode (e.g. the rib of the first electrode) and/or the fifth electrode (e.g. the rib of the fifth electrode), respectively.

In general, the first electrode, the fourth electrode, and/or the fifth electrode may be completely embedded within the first isolating portion and/or the second isolating portion, respectively, so that the intensity of the emitted microwave energy is reduced by the portion of the respective first isolating portion and/or the second isolating portion covering the respective electrodes by 5%, 10%, 15, 20%, or 25% compared to an intensity of the microwave energy that is emitted by the respective electrodes which are exposed at the respective first and second surfaces. So, the first isolating portion and/or the second isolating portion may a have thickness over the respective electrodes that attenuates the intensity of the emitted microwave energy by 5%, 10%, 15, 20%, or 25%.

Reasons for covering the first electrode, the fourth electrode, and/or the fifth electrode can be (i) to reduce the intensity of the microwave energy (i.e. not to reduce the actual quantity of power/energy transmitted to the tissue) and (ii) to provide a smooth and non-stick contact surface for the tissue. What this feature avoids can avoid is (i) energy being deployed into too small and focussed area of the tissue (e.g. the energy transmission can be diffused to a certain degree) and (ii) tissue heated by the microwave radiation (sometimes called cooked or charred tissue) from sticking to the metal/material of the electrodes/focal source of the energy transmission.

In an optional embodiment, the ribs of the first electrode and the fifth electrode are spaced away from each along a direction perpendicular to the longitudinal direction in the closed position.

The ribs of the first electrode and the fifth electrode may each extend parallel to the longitudinal direction of the first jaw and the second jaw, respectively. However, unlike the optional embodiments previously described, the first electrode (e.g. the rib thereof) and/or the fifth electrode (e.g. the rib thereof) may not be arranged in the middle between the exposed sections of the second electrode and the third electrode, respectively. Instead, the line of extension of the first electrode (e.g. the rib thereof) and/or the fifth electrode (e.g. the rib thereof) may be offset to a middle line along the direction of extension of the first jaw and/or the second jaw, respectively. Further, the line of extension of the first electrode (e.g. the rib thereof) and/or the fifth electrode (e.g. the rib thereof) may be spaced away from each other in the closed position. For example, the first electrode and/or the fifth electrode may be rotationally symmetric, e.g. around the middle lines on the first surface and the second surface in the closed position.

Optionally, the first jaw and the second jaw rotate in the same plane. In other words, an axis of rotation of the first jaw may be parallel or identical to an axis of rotation of the second jaw. During rotating or movement of the first jaw and the second jaw from the open position to the closed position, the first electrode may be moved along a plane that is parallel to a plane along with the fifth electrode is moved. In the closed position, the first electrode and the second electrode are closest to each other but spaced away from each other by the distance of these planes.

The second electrode may be completely covered by the first isolating portion and/or the third electrode may be completely covered by the second isolating portion. Optionally, the second electrode and/or the third electrode may have an L-shape in a cross-sectional view. This is different to the U-shaped previously discussed with the second electrode and the third electrode, respectively. So, the second electrode and/or the third electrode may include only one section that is close to (in case the second and/or third electrodes are completely covered by the first and/or second isolating portion, respectively) or exposed at the first surface and the second surface, respectively. In this case, this single section of the second electrode may effectively provide the ground electrode for the fifth electrode and/or this single section of the third electrode may effectively provide the ground electrode for the first electrode.

In an optional embodiment, the electrosurgical instrument further comprises a blade movable parallel and between the first electrode and the fifth electrode in the closed position.

The blade may be movable along the middle line of the first jaw (e.g. of the second electrode) and the second jaw (e.g. the third electrode) on the first surface and the second surface, respectively. The second electrode and/or the third electrode may include (each) include a recess or slot which may provide a rail for guiding and/or supporting the blade. The blade may have a size so that it is supported by the rails of the second electrode and the third electrode in the closed position. The first isolating portion and the second isolating portion may include a corresponding slit along which the blade can move. The first electrode may be arranged on a first side of blade and the fifth electrode may be arranged on the opposing second side of the blade. The axis of rotational symmetry of the first and fifth electrodes may lie on the cutting line of the blade.

The provision of the blade may allow for another possible mode of cutting tissue, namely by mechanical cutting using the blade. This can be done instead of or in addition to the radiofrequency energy cutting. The geometry of the first jaw and the second jaw in this configuration may allow a mechanically moving blade in the rail and/or slit. This action might allow, for example, simultaneous radiofrequency cutting or microwave sealing (from the first, second, and/or third electrodes) and mechanical cutting (from the blade) between the first jaw and the second jaw.

The blade may comprise a rigid element with a sharp edge adapted to slice biological tissue, e.g. a scalpel-type blade or the like. This type of blade is configured to perform a “cold” cut, which may be preferred because it carries a low risk of collateral thermal damage that is associated with other cutting techniques. However, the invention need not be limited to a cold cut blade. In other examples, the blade may comprise any one of: a bipolar radiofrequency cutting element, an ultrasound sonotrode, and a heatable wire element.

It has been found that radiofrequency cutting can be less effective on tissue that has previously been sealed (by microwave energy or other means of tissue sealing). So the mechanical cut by the blade can be made in cases where a RF is no longer possible or effective due to a dried-out tissue because of a previous microwave sealing of the tissue/vessel.

The blade may be movable with in the rail in the second isolating portion or within a groove in the second isolating portion. The blade may be exposed at and/or protrude from the second surface, e.g. the second isolating portion. The blade can define a cutting line by moving the blade. The cutting line can correspond to and/or be parallel to the gap on the first jaw and/or the slot/groove in the second isolating portion. The cutting line may be arranged between exposed sections of the second electrode or the third electrode. The cutting line may be in the middle of the sections of the second isolating portion that are exposed on the second surface.

The blade may be actuated by an actuation rod or control wire that can extend within the instrument shaft and is connected to the handle. The blade may have a cutting edge which is sufficiently sharp to mechanically cut tissue which is clamped between the first jaw in the second jaw in the closed position.

The blade may be in electrical connection with the second electrode and the third electrode which may act as ground electrodes for microwave sealing.

In an optional embodiment, the electrosurgical instrument further comprises a blade movable parallel the first electrode in the closed position, the blade being arranged on the second jaw and protruding from the second surface. Optionally, a gap is provided between the rib and the first isolating portion. Further optionally, the blade is movable in the gap in the closed position.

The blade may have the same optional features, embodiments, and/or characteristics as outlined above. Any optional differences are outlined below.

In one example, the blade may be slidable in the longitudinal direction between a retracted position in which it lies proximal to the pair of jaws and an extended position in which it lies within the region between the pair of jaws. It is desirable for the blade to slide into the region between the blade when they are in the closed position. The blade may be slidable along a longitudinally extending recessed groove formed in the pair of jaws, i.e. in each of the first jaw and the second jaw, so that it can contact tissue held between the jaws when the pair of jaws are closed.

The second jaw may include one or more guide rails along which, between which, or within which the blade is movable or slidable. The guide rail may be made from an electrically non-conductive material and can be in contact with the third electrode. For example, the guide rail may be supported by the third electrode and electrically isolates the blade from the third electrode. The second isolating portion may completely or partially cover the guide rail. The second isolating portion may include a slit above the guide rail in which the blade moves. The guide rail may be made from a ceramic material. Ceramics are more rigid than the elastically deformable material of the second isolating portion. So the guide rail can be additionally provided for enhancing the stability of the blade support which may not be feasible with the material of the second isolating portion.

As the blade protrudes from the second surface, the first jaw needs to include a slot or channel in the first isolating portion that runs parallel to the slit in the second isolating portion for receiving the blade in the closed position. In one example, this slot or channel is provided by the gap between the rib and a portion of the first isolating portion. For example, the rib extends parallel and offset to the guide rail in the closed position so that the blade is movable along a side surface of the rib. A portion of the first isolating portion facing the side surface of the rib is spaced away from the side surface of the rib for providing the gap. The blade may move between the side surface of the rip and the opposing portion of the first isolating portion. The blade may be in contact with the side surface of the rib and/or the opposing portion of the first isolating portion in the closed position when moving along the guide rail/cutting line. This may support the blade against forces perpendicular to the cutting line.

In another example, the above-described slot or channel is provided by a slot or open channel in the first isolating portion. So, to opposing sides of the blade may face different sections of the first isolating portion. This is in contrast to the above-described embodiment in which one side of the blade faces the rib and the other side of the blade faces a portion of the first isolating portion.

In an optional embodiment, the blade is electrically isolated from the third electrode and spatially offset from the first electrode in the closed position. Optionally, the blade is electrically connected to the transmission line to provide a counter electrode to the first electrode for radiofrequency cutting between the first electrode and the blade.

The electrical isolation of the blade with respect to the third electrode may be provided by the guide rail(s) being made from an electrically insulating material as described above. Further, the guide rail is arranged such that the blade is offset to the first electrode or the rib thereof perpendicular to the cutting line/extension of the guide rail in the closed position. Thus, when moving the blade along the guide rail, the blade is not in contact with the first electrode or the rib of the first electrode. A portion of the first isolating portion or air may be provided between the blade and the first electrode.

In this case, the blade may act as a counter electrode to the first electrode for providing radiofrequency cutting between the first electrode and the blade. In this case, the blade not only provides a cold cut but also a radiofrequency cut. To this end, the blade is electrically connected to the transmission line. For example the transmission line includes two wires for providing the energy for the radiofrequency cutting wherein the first wire is electrically connected to the first electrode and the second wire is electrically connected to the blade. Optionally, the push rod for actuating the blade may also provide the electrical connection for the radiofrequency cutting.

In an optional embodiment, the electrosurgical instrument further comprises a second isolating portion arranged in the channel of the third electrode.

The second isolating portion may include the same features, characteristic, and/or optional embodiments as the first isolating portion. For example, the second isolating portion is attached to the third electrode, for example by adhesion and/or by form fit (positive fit, positive mechanical engagement, or positive interlocking). Optionally, one or more protrusions and/or one or more recesses are provided in the channel of the third electrode which provide an undercut for the second isolating portion. A form fit may be used for attaching the second isolating portion to the third electrode in case silicone is used for the second isolating portion because silicone is non-stick so that it is less suitable for adhesion with the third electrode. For example, the second isolating portion in a fluid state is poured into the channel of the third electrode and then cured or hardened so that it interlocks with the one or more protrusions and/or one or more recesses provided in the channel of the third electrode.

In an optional embodiment, the electrosurgical instrument further comprises a fourth electrode for emitting microwave and/or radiofrequency energy and arranged on the first jaw and/or a fifth electrode for emitting microwave and/or radiofrequency energy and arranged on the second jaw. Optionally, the fourth electrode and/or the fifth electrode each have a U-shape in a cross-sectional view.

In an optional embodiment, end faces of the fourth electrode are at least partially exposed on the first surface, wherein optionally the exposed sections of the fourth electrode are offset to the first electrode. Alternatively or additionally, end faces of the fifth electrode may be at least partially exposed on the second surface, wherein optionally the exposed sections of the fifth electrode are offset to the first electrode in the closed position.

For example, portions of the second electrode, the third electrode, the fourth electrode, and/or the fifth electrode are plate-shaped and respectively include two end faces. Optionally the end faces form sections of the second electrode, the third electrode, the fourth electrode, and/or the fifth electrode that are exposed on the first surface and the second surface, respectively. The portions of the second electrode and the fourth electrode are U-shaped or V-shaped in a cross-sectional view of the first jaw. Further, the portions of the third electrode and the fifth electrode are U-shaped or V-shaped in a cross-sectional view of the second jaw.

For example, the portions of the second electrode and/or the fourth electrode that extend along sections of the first jaw where the first electrode is provided may be plate-shaped. Other portions of the second electrode and/or the fourth electrode may have different configurations. For example, proximal and/or distal end portions of the second electrode and/or the fourth electrode may have shapes that deviate from a plate shape. This may be provided for forming distal and/or proximal end portions of the first jaw. End faces of the plate-shaped portions of the first electrode, the second electrode and/or the fourth electrode can be the exposed sections on the first surface.

The portions of the third electrode and/or the fifth electrode that extend along sections of the second jaw which oppose the first electrode in the closed position may be plate-shaped. Other portions of the third electrode and/or the fifth electrode may have different configurations. For example, proximal and/or distal end portions of the third electrode and/or the fifth electrode may have shapes that deviate from a plate shape. This may be provided for forming distal and/or proximal end portions of the second jaw. End faces of the plate-shaped portions of the third electrode and/or the fifth electrode can be in the exposed sections on the second surface. The third electrode may be mirror-symmetric to the second electrode and/or the fifth electrode may be mirror-symmetric to the fourth electrode.

The plate-shaped portions of the second electrode, the third electrode, the fourth electrode, and/or the fifth electrode may have a shape of the letter U, V, or variations thereof in a cross-sectional view of the first jaw. For example, the first electrode and/or the first isolating portion may be arranged within the shape defined by the U-shape or V-shape in a cross-sectional view.

The first surface may include the exposed sections of the first electrode, the exposed sections of the second electrode, the exposed sections of the first isolating portion, and/or the exposed sections of the fourth electrode. The exposed sections of the first electrode, the exposed sections of the second electrode, the exposed sections of the first isolating portion, and/or the exposed sections of the fourth electrode may be provided by the respective end faces. The first electrode, the second electrode, and/or the fourth electrode are arranged within and/or on the first jaw. The first electrode, the second electrode, and/or the fourth electrode are made from an electrically conductive material, such as metal, and may be connected to an inner conductor and an outer conductor of the coaxial cable, respectively.

The second surface may include the exposed sections of the third electrode and/or the exposed sections of the fifth electrode. The third electrode and/or the fifth electrode are arranged within and/or on the second jaw. The third electrode and/or the fifth electrode are made from an electrically conductive material, such as metal, and may be connected to an inner conductor and an outer conductor of the coaxial cable, respectively.

The fourth electrode and the fifth electrode may be electrically connected to each other and/or to the inner conductor of the coaxial cable. Further, the second electrode and the third electrode may be electrically connected to each other and/or to the outer conductor of the coaxial cable. So, the fourth electrode and the fifth electrode can be active electrodes for emitting microwave energy for microwave sealing and the second electrode and the third electrode can be ground electrodes for confining the emitted microwave energy.

In an optional embodiment, the exposed sections of the fourth electrode are arranged between sections of the second electrode that are exposed on the first surface and/or the exposed sections of the fourth electrode are arranged between sections of the third electrode that are exposed on the second surface.

The exposed sections of the second electrode and/or the fourth electrode may extend as lines on the first surface. So the exposed sections of the fourth electrode may be arranged between the exposed sections of the second electrode. So, in atop view on the first surface, the exposed sections on the first surface are along a line perpendicular to the longitudinal direction of the first jaw: end face of the second electrode, end face of the fourth electrode, end face of the fourth electrode, and end face of the second electrode.

Similarly, the exposed sections of the third electrode and/or the fifth electrode may extend as lines on the second surface. The exposed sections of the fifth electrode may be arranged between the exposed sections of the third electrode. So, in a top view on the second surface, the exposed sections on the second surface are along a line perpendicular to the longitudinal direction of the second jaw: end face of the third electrode, end face of the fifth electrode, end face of the fifth electrode, and end face of the third electrode.

The first electrode may also extend as a line on the first surface (which can form the cutting line). In an embodiment without the fourth and/or fifth electrode, the first electrode and/or the exposed sections of the second electrode define a sealing area. The second electrode may be electrically isolated from the fourth electrode by the first isolating portion. The third electrode may be electrically isolated from the fifth electrode by the second isolating portion.

The exposed sections of the second electrode, the third electrode, the fourth electrode, and/or the fifth electrode are separated from each other on the respective surfaces. The exposed sections of the second electrode and/or the fourth electrode may form (straight) lines between which the first electrode is positioned. Exposed sections of the first isolating portion may be arranged between the (straight) lines of the first electrode and the exposed sections of the second electrode and/or between the (straight) lines of the second electrode and the exposed sections of the third electrode. So, in atop view on the first surface, the first electrode may be between the exposed sections or end faces of the fourth electrode, all of which are between the exposed sections or end faces of the second electrode. Similarly, in a top view on the second surface, the exposed sections or end faces of the fifth electrode may be parallel to each other and between the exposed sections or end faces of the third electrode.

A sealing area is defined as being configured to emit microwave energy at the first electrode and the exposed sections of the second electrode in case no fourth electrode is provided. For example, the first electrode may be considered an active electrode and the exposed section of the second electrode may be considered a return electrode for radiofrequency cutting. Further, the first electrode and the exposed sections of the second electrode may be a dipole antenna for radiating microwave electromagnetic energy. In this case, RF cutting can be provided also between the first electrode and the second electrode. So, the sealing area of the microwave sealing and the area of RF cutting (substantially) overlap or are almost identical.

In case the fourth electrode is provided, a sealing area is defined as being configured to emit microwave energy at the exposed sections of the fourth electrode and the exposed sections of the second electrode. For example, the fourth electrode may be considered an active electrode and the exposed section of the second electrode may be considered a return electrode for radiofrequency cutting. Further, the exposed sections of the fourth electrode and the exposed sections of the second electrode may be a dipole antenna for radiating microwave electromagnetic energy. The fifth and third electrode may define the same sealing area as the fourth and second electrodes but from a different side of the tissue grasped between the first jaw and the second jaw.

The respective sealing areas are configured to emit microwave energy to tissue that is close to or in contact with the exposed sections of the electrodes involved in microwave sealing. The exposed sections of the first electrode, the second electrode, the third electrode, the fourth electrode, and/or the fifth electrode are arranged such that tissue that is clamped and/or grasped between first surface and the second surface in the closed position is in contact with the exposed sections of the first electrode, the second electrode, the third electrode, the fourth electrode, and/or the fifth electrode—in other words in contact with the sealing area. This means that the sealing area may have a several functionalities. They can clamp or grasp tissue (with the second surface being the counterpart), emit microwave energy, and/or emit radiofrequency energy.

The greatest intensity of the emitted microwave energy is achieved in a portion of the tissue that is in contact with or directly above the exposed sections of the active electrodes. In particular, the intensity of the emitted microwave energy is highest at respective edges or corners of the exposed sections of the active electrodes and the exposed sections of the return electrodes.

In case the fourth and/or fifth electrodes are provided the sealing areas are between the exposed sections of the fourth electrode and second electrode and/or between the exposed sections of the fifth electrode and third electrode. The radiofrequency cutting can be provided between the first electrode and the exposed sections of the fourth electrode. In this case, the fourth electrode has a double-functionality as active electrode for microwave sealing and counter electrode for radiofrequency cutting. As the first electrode is arranged between the exposed sections of the fourth electrodes, the sealing area is offset to and/or does not overlap with the radiofrequency cutting. This reduces or eliminates the risk that the microwave sealing completely dries out the tissue at the first electrode so that RF cutting is no longer possible.

The U-or V-shape of the fourth electrode and/or the fifth electrode can provide a form fit with the first isolating portion and/or the second isolation portion, respectively. The fourth electrode and/or the fifth electrode are attached to and/or supported by the first isolating portion and/or the second isolation portion, respectively, for example by means of their shape.

In an optional embodiment, the electrosurgical instrument further comprises a sixth electrode which extends parallel to the first electrode on the first jaw for providing radiofrequency cutting between the first electrode and the sixth electrode.

The sixth electrode may include the same optional characteristics, features, and/or embodiments as the first electrode. For example, the sixth electrode may also be a floating electrode that is attached to and supported by the first isolating portion so that the sixth electrode is movable relative to the second/fourth electrode upon an application of pressure on the sixth electrode.

The sixth electrode may run parallel to the first electrode along the longitudinal direction of the first jaw. For example, a gap between the first electrode and the sixth electrode is constant along the direction of extension of the first jaw. The sixth electrode may act as a counter electrode for the first electrode during radiofrequency cutting.

In the absence of the fourth electrode and/or the fifth electrode, the first electrode and sixth electrode may act as active electrodes for microwave sealing and the second electrode and/or the third electrode may act as a return electrode for the microwave sealing. In this case, the sealing area as defined between the exposed section of the second electrode and the first electrode and the sealing area is defined between the exposed section of the second electrode and the sixth electrode are spatially offset from the area of radiofrequency cutting which is between the first electrode and the sixth electrode. The first electrode and the sixth electrode may be arranged in the middle between the exposed sections of the second electrode. As described above, the spatial separation of microwave sealing and radiofrequency cutting reduces the risk of completely drying out tissue during microwave sealing so that radiofrequency cutting becomes impossible.

The exposed sections of first electrode, the second electrode, the third electrode, the fourth electrode, the fifth electrode and/or the sixth electrode may be used for measuring current between any two of the electrodes.

In an optional embodiment, the fifth electrode is completely embedded within the second isolating portion and/or the fourth electrode is completely embedded within the first isolating portion.

In this embodiment, the fourth electrode and/or the fifth electrode may have the same configuration as described above except that the end faces are not exposed at the first surface and the second surface respectively. Rather, the end faces of the first electrode and/or the second electrode are also covered by the first isolating portion and the second isolating portion, respectively. In other words, the fourth electrode and/or the fifth electrode is completely buried or completely embedded within the first isolating portion and the second isolating portion, respectively. This reduces the risk that tissue sticks to the end faces of the fourth electrode and/or the fifth electrode because the respective end faces are covered by the non-stick material of the first isolating portion. At the same time, the fourth electrode and/or the fifth electrode may act as active electrodes for the emission of microwave energy because the tissue does need not to be in direct contact with the fourth electrode and/or the fifth electrode for microwave sealing.

In an optional embodiment, the second electrode is electrically connected to the third electrode and the fourth electrode is electrically connected to the fifth electrode so that microwave energy is generated between the pair of second electrode and third electrode and the pair of fourth electrode and fifth electrode.

This embodiment refers to the presence of the fourth electrode and/or the fifth electrode which provides the sealing areas for microwave sealing as described above. However, a difference to the above-described electrosurgical instrument only including a first electrode compared to the embodiment having the first and the sixth electrodes is that there is a further separation between microwave sealing and radiofrequency cutting. The presence of the sixth electrode ensures that the radiofrequency cutting is achieved between the first electrode and the sixth electrode both of which are spatially offset to the exposed sections of the fourth electrode providing the edge of the sealing areas for micro wave sealing. In other words, there are gaps extending perpendicular to the longitudinal direction of the first jaw between the area of radiofrequency cutting and the microwave sealing areas on both sides of the area of radiofrequency cutting. If only the first electrode is provided (i.e. in the absence of the sixth electrode), there is no gap between the area of radiofrequency cutting and microwave sealing since the fourth electrode has to double functionality as counter electrode for radiofrequency cutting and active electrode for microwave sealing. So, the presence of the sixth electrode provides a clear separation between radiofrequency cutting (for which the first electrode and a sixth electrode are provided) and microwave sealing (for which the fourth electrode and the second electrode are provided)—so no electrode has a double functionality in this embodiment. This may also simplify the electrical connection of the respective electrodes to the transmission line since each electrode may need to be connected to a single line/wire of the transmission line.

In an optional embodiment, the first electrode and the sixth electrode are made from an electrically conductive material and/or the sixth electrode is mirror symmetric to the first electrode.

In an embodiment, both the first electrode and the sixth electrode can be floating electrodes as described above. The sixth electrode may be mirror symmetric along a line extending along the longitudinal direction of the first jaw and in the middle between the first electrode and the second electrode.

In an optional embodiment, the electrosurgical instrument further comprises an electrode support made form an electrically isolating material. Optionally, the electrode support includes a support base plate and a support rib arranged on the support base plate for forming a T-shape in a cross-sectional view of the first jaw. Further optionally, the first electrode and the sixth electrode each include a strip made from an electrically conductive material and arranged on opposing sides of the support rib.

The support base plate and/or the support rib may each have an elongate shape and can be made from a plate or strip of electrically non-conductive material such as ceramic. The support base plate can be (permanently) attached to the support rib, for example by adhesion. Alternatively, the support base plate and the support rib are a unitary component.

Most of the support structure is buried in or covered by the first isolating portion. A section or portion of the support structure may be exposed at the first surface or protrude from the first isolating portion. The support base plate may be provided for interlocking the support structure with the first isolating portion. For example, the support base plate protrudes on one or both sides from the support rib. For example, a width of the support base plate is larger than a thickness of the support rib wherein the width and the thickness are measured in the same direction in an assembled state of the support structure. The support base plate may extend perpendicular to the support rib. The support base plate may be attached to the support rib at an end of the support rib which is opposite to the end of the support rib that is exposed at the first surface. The support base plate may be fully embedded in the first isolating portion.

The provision of the support base plate may be a means for interlocking the support structure with the first isolating portion and/or may be provided as an alternative or in addition to one or more through-holes and/or protrusions in/on the support structure for providing an undercut. As described above, the first isolating portion can be formed by pouring the first isolating portion (in a fluid state) in the channel of the second electrode while holding the support structure in its desired position (e.g. relative to the second electrode).

The support structure is provided in one embodiment for supporting the first electrode and/or the sixth electrode. In this case, the first electrode and/or the sixth electrode may be completely made from an electrically conductive material that are not directly attached to and supported by the first isolating portion. Rather, in this embodiment, the first electrode and/or the sixth electrode are indirectly attached by and connected to the first isolating portion, namely via the support structure. In this case, the first electrode and/or the sixth electrode may not be attached by interlocking as described above, but by other means such as adhesion and the like. The first electrode and/or the sixth electrode may be attached to a portion or section of the support structure that is exposed so that the first electrode and/or the sixth electrode may not be (partially) buried in a first isolating portion.

Optionally, the first electrode and/or the sixth electrode may each include or are completely made of a strip made from elastically conductive material such as metal or a metal alloy. As described above, the first electrode and the sixth electrode need to be separated for providing radiofrequency cutting. This can be achieved by arranging to strip of the first electrode on one side of the support rib and the strip of the sixth electrode on the other side of the support rib. So, the thickness of the support rib defines the distance between the first electrode and the sixth electrode. Further, the support rib provides the electrical isolation between the first electrode and the sixth electrode. The strip of the first electrode and/or the strip of the sixth electrode may each be attached to the support structure by means of adhesion or the like.

In an optional embodiment, the support base plate is in contact with the fourth electrode.

For example, the support base plate may be in (direct) contact with a bottom portion of the fourth electrode that is fully buried in the first isolating portion.

In an optional embodiment, the fifth electrode interlocks with the second isolating portion and is exposed on the second surface so that the fifth electrode contacts the first electrode in the closed position.

In this embodiment, the fifth electrode may not have a U-shape in a cross-sectional view but may have a T-shape or is a bar/rod. The fifth electrode may be mirror symmetric to the first electrode in the closed position. The fifth electrode may protrude from the second isolating portion and/or may be in contact with the first electrode in a closed position. The fifth electrode may have to same characteristics, features and/or optional embodiments as the first electrode for interlocking with the second isolating portion. For example, the fifth electrode may also be a floating electrode similar to the first electrode such that both the first electrode and the fifth electrode move when tissue is grasped between the first jaw and the second jaw in the closed position.

In an embodiment, the fifth electrode may be a counter electrode to the first electrode for radiofrequency cutting. In this case, the radiofrequency cutting is facilitated between the first jaw and the second jaw. Further, the first electrode and/or the fifth electrode may be an active electrode for microwave sealing; the second electrode and/or the third electrode may act as return electrodes.

In an optional embodiment, the electrosurgical instrument further comprises a liquid output and a liquid feed for supplying liquid to the liquid output. Optionally, the liquid output is arranged between the first electrode and the first isolating portion for wetting the first surface at the first electrode.

The liquid output and the liquid feed are provided for re-wetting tissue or maintaining wetness/moisture of the tissue that is grasped between the first jaw and the second jaw. As outlined above, microwave sealing of the tissue results in a decrease of the moisture or liquid within the tissue. If the tissue is completely dried out, radiofrequency cutting is no longer possible. To avoid a complete drying out of the tissue during or after micro wave sealing, the liquid output and a liquid feed provide liquid at the first surface and/or the second surface so that the tissue grasped between the first jaw and the second jaw is either re-wetted (e.g. after being completely dried out during microwave sealing) or is constantly wetted during microwave sealing so that the tissue does not completely dry out. So, the liquid output is configured to provide liquid at the first surface and/or the second surface. In other words, the liquid output is configured to wet tissue that is grasped between the first jaw and the second jaw. Further, the liquid output may be configured to supply the liquid during microwave sealing and/or radiofrequency cutting.

The liquid output may include one or more liquid outlets on the first surface and/or the second surface. The liquid outlets may extend along or are distributed along the longitudinal extension of the first jaw and/or the second jaw. For example, the liquid outlets supply liquid along the first electrode, the second electrode, the third electrode, the fourth electrode, the fifth electrode, and/or the sixth electrode. The liquid outlets may be in contact with, adjacent to, and/or between the respective electrodes on the first surface and/or the second surface.

The liquid feed may include one or more pipes and/or hoses which can extend through the instrument shaft, along the first jaw and/or the second jaw to the liquid output. A first end of the liquid feed may extend beyond the proximal end of the instrument shaft and can be connected to a liquid reservoir. The liquid feed may further include a channel and/or passage within the first jaw and/or the second jaw for feeding or supplying liquid along the first jaw and/or the second jaw. The channel and/or passage of the liquid feed may be an integral structure of the first jaw and/or the second jaw. For example, the channel and/or passage of the liquid feed may be a cavity within the second electrode and/or the third electrode. The pipes and/or hoses of the liquid feed may be connected to the channel and/or passage of the liquid feed.

The liquid reservoir may store the liquid that is supplied to the liquid output. The liquid may be a buffer solution, saline solution, sterilised water, and/or any other type of nontoxic liquid that can be supplied within the cavity of a body without harming tissue. The liquid reservoir may be an IV drip (a container containing liquid for intravenous (IV) therapy). In this case, gravity provides the pressure for pushing the liquid along the liquid output and the liquid feed. Alternatively or additionally, a pressurised liquid reservoir may be used, e.g. a pressurized (saline) canister. Further, the liquid reservoir can be coupled to a liquid pump for pumping the liquid along the liquid feed and a liquid output.

Further, a valve for controlling the flow within the liquid feed may be attached to liquid feed. The valve may be a solenoid pinch valve which can open, partially open, and close the liquid feed so that liquid, a reduced amount of liquid, and no liquid, respectively, can flow along the liquid feed.

The liquid output may optionally be arranged or located close to or adjacent to the electrodes that provide the radiofrequency cutting, preferably the active electrode for radiofrequency cutting. As an example, the liquid output may be arranged at the first electrode if the first electrode participates in radiofrequency cutting. In one embodiment, the liquid output and/or the liquid outlet(s) are positioned between the first electrode and the first isolating portion. For example, the liquid output includes one or more channels that connect the liquid feed to the liquid outlets which are arranged at the first surface between the first electrode and the first isolating portion. The channel(s) of the liquid output may be provided or defined by the first electrode and/or the first isolating portion. In other words, the outer walls of the channels of the liquid output are defined by a cavity, a recess, and/or a slot within the first electrode and/or the first isolating portion. As an example, the first isolating portion and the first electrode commonly define a channel of the liquid output. The liquid outlet may be an open end of the channel of the liquid output.

The diameter of the liquid outlet and/or the channel of the liquid output may be chosen that the liquid is sucked by capillary forces from the liquid feed to the first surface and/or the second surface. The small diameter of the liquid output (e.g. the liquid outlet and/or the channel) may limit the amount of liquid that is supplied and/or provides only liquid in case of the absence of liquid in the tissue. So the capillary forces can be used to control the amount of liquid that is supplied to the tissue grasped between first jaw and the second jaw.

In an optional embodiment, the liquid output includes a plurality of liquid outlets along the first electrode and/or a single liquid outlet extending along a section of the first electrode.

The plurality of liquid outlets may be evenly distributed along the first electrode and/or any other electrode for providing an even supply of liquid along the longitudinal extension of the first jaw and/or the second jaw. This can ensure that the tissue grasped between the first jaw and the second jaw is evenly wetted along the area where the radiofrequency cut is provided. Alternatively or additionally, the liquid outlet is a single elongate outlet that extends along the longing to the direction of the first jaw and/or the second jaw. In this case, the liquid outlet may be a slit or slot on the first surface and/or the second surface.

According to another aspect of the present invention, there is provided an electrosurgical instrument for sealing and/or cutting tissue which comprises an instrument shaft, a first jaw, a second jaw, a first electrode, a second electrode, a first isolating portion, a liquid output, and a liquid feed for supplying liquid to the liquid output. The instrument shaft comprises a (coaxial) transmission line for conveying microwave electromagnetic energy and radiofrequency electromagnetic energy. The first jaw is attached to the instrument shaft and includes a first surface. The second jaw is attached to the instrument shaft and includes a second surface. The first and/or second electrodes are configured to emit micro wave and/or radiofrequency electromagnetic energy. The first isolating portion electrically isolates the first electrode from the second electrode, at least in a closed position. The first jaw and the second jaw can be moved between an open position, in which the tissue can be inserted between the first surface and the second surface, and the closed position, in which the first and second surfaces are brought together to clamp, hold, and/or grasp tissue therebetween. The first electrode can be exposed on the first isolating portion. The liquid output is optionally arranged between the first electrode and the first isolating portion for wetting the first surface at the first electrode

The electrosurgical instrument of this aspect may include the same features, optional embodiments, and/or characteristics as described in connection with the other aspects.

In an optional embodiment, a first face of the first isolating portion, the second electrode, and/or the fourth electrode and a second face of the first isolating portion, the second electrode, and/or the fourth electrode are arranged on opposing sides of the first electrode and/or the sixth electrode, wherein the first face and the second face define an angle of less than 180° with respect to each other.

For example, the first face and the second face define an angle in the range of 100° to 180°, e.g. 120°, 150°, or 170°. The inclined configuration of the first face and the second face increases the exposure of the first electrode so that the pressure of the first electrode in the closed position on the clamped tissue is further increased.

The first face may be (or solely include) a section of the first isolating portion that is exposed on the first surface on one side of the first electrode and/or sixth electrode, and the second face may be (or solely include) a section of the first isolating portion that is exposed on the first surface on another side of the first electrode and/or sixth electrode. In this embodiment, the first face and the second face do not include sections of the second electrode that are exposed on the first surface.

The exposed section of the first isolating portion (i.e. the first face and/or the second face) may be flat or planar. The first electrode may be arranged at that line where the first face and the second face would meet. Further, the first face and the second face may be symmetrical about the first electrode and/or the sixth electrode. Sections of the second electrode that are exposed on the first surface may be flush with the first face and/or the second face, respectively.

Alternatively, the first face and the second face may include sections of the first isolating portion, the second electrode, and/or the fourth electrode that are exposed on the first surface. Further alternatively, the first face and the second face may only include sections of the second electrode and/or the fourth electrode that are exposed on the first surface. In this embodiment, the first face and the second face do not include sections of the first isolating portion that are exposed on the first surface.

In an optional embodiment, the second surface includes a third face and a fourth face. Optionally, the first face is parallel to the third face and the second face is parallel to the fourth face. Alternatively, the angle defined by the first face and the second face may be smaller than an angle defined by the third face and the fourth face. Further alternatively, the third face and the fourth face may define an angle of 180° with each other.

The third face and the fourth face may be surface areas of the second surface which may be equivalents to the first face and the second face, respectively. For example, the third face has the same surface area as the first face, and the fourth face has the same surface area as the second face. In the closed position, the first face and the second face partially or completely contact the third face and the fourth face, respectively. In the closed position, the first electrode may contact a line on the second surface which separates the third face from the fourth face.

For example, the third face and the fourth face define an angle in the range of 100° to 180°, e.g. 120°, 150°, or 170°.

The third face may include sections of the second isolating portion, the third electrode, and/or the fifth electrode that are exposed on the second surface, and the fourth face may include sections of the second isolating portion, the third electrode, and/or the fifth electrode that are exposed on the second surface.

The exposed section of the second isolating portion, the third electrode and/or the fifth electrode (i.e. the third face and/or the fourth face) may be flat. Further, the third face and the fourth face may be symmetrical to each other. Sections of the third electrode and/or the fifth electrode that are exposed on the second surface may be flush with the sections of the second isolating portion that are exposed on the second surface, respectively.

The first, second, third, and/or fourth faces may each be flat or straight surfaces.

In the embodiment of the parallel arrangement of the first face to the third face and of the second face to the fourth face, the first face may completely contact or cover the third face in a closed position and the second face may completely contact or cover the fourth face in the closed position. Thus, pressure on the tissue between the first face and the third face as well as the second face and the fourth face in the closed position may be constant along the respective surface areas. The pressure on the tissue that is applied by the first electrode is increased compared to the pressure applied by the first to fourth face due to the parallel arrangement of the respective faces.

In the embodiment in which the angle defined by the first face and the second face is smaller than the angle defined by the third face and fourth face, the pressure applied to the tissue by the respective faces decreases from the first electrode outwards (e.g. perpendicular to the longitudinal direction of the first jaw) since a gap between the first face and the third face as well as between the second face and the fourth face in the closed position increases with increased distance from the first electrode. In this configuration, pressure on the tissue can be increased or concentrated on the area of the first electrode or, in other words, in the area where radiofrequency is delivered.

In the embodiment in which the third face and the fourth face define an angle of 180°, the third face and the fourth face may define a flat or straight surface. For example, the second surface may be flat. Here again, the pressure applied on the tissue by the respective faces decreases from the first electrode outwards since a gap between the first face and the third face as well as between the second face and the fourth face in the closed position increases with increased distance from the first electrode.

In an optional embodiment, the second isolating portion includes a lumen and the lumen is configured to be deformed in the closed position, wherein optionally the electrosurgical instrument comprises a blocking element which is insertable into the lumen for filling the lumen.

The second isolating portion may include a flexible tube or hose. The lumen may be a channel or passage extending parallel to the first electrode in the closed position. The lumen is arranged close to the second surface such that the first electrode deforms the second isolating portion in the closed position, in particular, compresses the lumen. So in the closed position, the volume of the lumen is reduced due to the pressure applied by the first electrode compared to the open position. The lumen may be elastically deformable. The lumen may be closed or open at the distal end of the second jaw. The compression of the lumen allows to reduce the pressure that is applied on the tissue in the closed position since the deformation of the lumen results in a decrease of the pressure on the tissue.

The blocking element may include a wire, rod, and/or any other elongate body that are configured to be inserted into the lumen. The blocking element may be made from (stainless) steel or Nitinol (NiTi). The blocking element may be inserted by distally advancing the blocking element from a proximal end of the lumen to a distal end of the lumen. The blocking element may be activated by a control wire or actuation rod or forms a unitary component with the control wire or actuation rod.

If the blocking element is inserted into the lumen, the blocking element substantially fills the lumen so that the lumen can no longer be deformed upon application of pressure. The blocking element may be used for increasing the pressure on the tissue in the closed position. For example, for microwave sealing, the blocking element is not inserted into the lumen and the electrosurgical instrument is brought into the closed position. As such, the first electrode presses against the second isolating portion and reduces the volume of the lumen. This is helpful for releasing the pressure on the tissue between the second isolating portion and the first electrode. Prior to radio frequency cutting, the blocking element is inserted into the lumen so that the lumen is expanded from the compressed configuration so that additional pressure is applied to the tissue grasped between the first electrode and the second isolating portion.

In an optional embodiment, the second isolating portion forms the third face and the fourth face. Optionally, the second isolating portion protrudes from the third electrode. Further, optionally only the second isolating portion is in contact with the first surface in the closed position.

Only the second isolating portion may form the third face and the fourth face. In this case, the third electrode may also be exposed at the second surface. However, the exposed sections of the third electrode are offset away from the first surface in the closed position so that the second isolating portion first comes into contact with the tissue or the first surface when moving the first jaw and the second jaw towards to the closed position. It is possible that only the second isolating portion is in contact with tissue or the first surface in the closed position, for example if the offset between the first and second faces and the exposed sections of the third electrode is large.

In this embodiment, the second isolating portion can be made from a soft flexible material such as silicone or silicone-based material. Due to the protrusion of the second isolating portion from the third electrode, pressure that is applied to the tissue in the closed position can be balanced and/or reduced.

In an optional embodiment, the second isolating portion includes an elongate recess or open channel for receiving the first electrode and/or the sixth electrode in the closed position. In this way, the pressure on the tissue between the first electrode and/or the sixth electrode and the second surface can be reduced in the closed position, especially when the first electrode and/or the sixth electrode protrude from the first isolating portion.

In an optional embodiment, the exposed sections of the second electrode, the fourth electrode, and/or the first electrode at least partially extend parallel to each other. Optionally, the first electrode is arranged between the exposed sections of the second electrode and/or the fourth electrode.

The exposed sections of the second electrode and/or the fourth electrode completely or partially extend parallel to each other on the first surface, respectively. For example, the exposed sections are straight which are connected by a connecting section-thus forming a loop. Other shapes of the loops are possible. The first electrode may completely or partially extend parallel to the exposed section of the second electrode. In case of a loop, the second electrode forms a single section that is exposed on the first surface.

In an optional embodiment, the first jaw includes a distal end face, wherein the first electrode, the sixth electrode, and/or the first isolating portion are exposed on the distal end face.

The first and sixth electrodes can operate to provide a localised cut in a biological vessel/tissue gripped between the first and second jaws. Further, the first electrode and the sixth electrode can be further used for radiofrequency cutting at the distal end face. In a position of the first and second jaws in which the second jaw does not cover the section of the first electrode and the sixth electrode exposed on the distal end face (e.g. the open position), the sections of the first electrode and the sixth electrode that are exposed on the distal end face can act as an active electrode and a return electrode for radiofrequency cutting. This may allow fine cutting at the distal end face of the electrosurgical instrument, for example, for cutting a hole into tissue so that the electrosurgical instrument can be further advanced into and/or through the tissue, for example, as part of a tunnelling procedure. Further, the first and second electrodes exposed on the distal end face can be used to cut fine and/or small sections of tissue that are clamped or grasped between the distal end face and the second jaw. Thus, tissue can be “nibbled”.

The first surface includes the first electrode and the sixth electrode. Other sections of the first electrode and the sixth electrode are exposed on the distal end face. The first electrode and/or the sixth electrode may be connected to an inner conductor and an outer conductor of the coaxial cable, respectively.

The sections of first electrode and the sixth electrode that are exposed on the distal end face are spaced apart from each other, for example by an air gap or the first isolating portion (e.g. an exposed section thereof).

The first surface and the distal end face may form a continuous surface of the first jaw. The first surface and the distal end face may be inclined relative to each other. For example, an angle between a plane defined by the first surface and a plane defined by the distal end face may form an angle between 10° to 90°, optionally 45°, 60°, or 90°. The distal end face, the first surface, and an outer surface of the second electrode may define an outer surface of the first jaw. In this configuration, the distal end face is a side surface while the first surface and the outer surface of the second electrode are surface that extends along the longitudinal direction of the first jaw. In other words, the distal end face extends to transverse to the longitudinal direction of the first jaw. The distal end face may be a surface of the first jaw that is arranged at a distal-most position of the first jaw. In other words, when moving the first jaw along the longitudinal direction of the first jaw towards the tissue, the distal end face firstly and/or solely contacts the tissue.

The distal end face may be straight/flat. Alternatively, the distal end face may be curved. The sections of the first electrode, the sixth electrode, and the first isolating portion that are exposed at the distal end face may be flush with respect to each other or define a flat surface. Alternatively, the sections of the first electrode and/or the sixth electrode that are exposed at the distal end face may protrude from the section of the first isolating portion that is exposed at the distal end face.

The second jaw (e.g. the third electrode) may partially or completely cover the first surface and/or a distal end face in the closed position of the first jaw and the second jaw. In any case, the second jaw (e.g. the third electrode) covers the section of the first electrode that is exposed at the distal end face and/or the section of the sixth electrode that is exposed to the distal end face in a closed position. Stated differently, if the first jaw and the second jaw are brought together with no tissue therebetween, the second jaw covers and/or contacts the sections of the first electrode and/or the sixth electrode that are exposed on the first surface and/or the distal end face. In this way, the second jaw may function to shield the first and/or sixth electrode(s) when the jaws are closed, for example, to avoid unintentionally treating tissue whilst the instrument is moved into position at a treatment site.

The sections of the first electrode and the sixth electrode exposed at a distal end face can define an active electrode and a return electrode, respectively, which can be used for cutting tissue at the distal end face. The first electrode and the sixth electrode can be connected to the transmission line which is configured to convey both microwave and radiofrequency energy.

In an optional embodiment, the second jaw includes an overhang portion protruding from the second surface. Optionally the overhang portion covers sections of the first electrode and/or the sixth electrode that are exposed on the distal end face in the closed position of the first jaw and the second jaw.

The overhang portion may be provided by the third electrode and may include a side surface which is continuous with the second surface. The second surface and the side surface may be inclined to each other. For example, an angle between a plane defined by the second surface and a plane defined by the side surface may form an angle between 10° to 90°, optionally 45°, 60°, or 90°. This angle may correspond to (e.g. be identical to) the angle defined by the first surface and the distal end face. In the closed position, the distal end face and side surface may extend parallel to each other and/or contact each other.

The overhang portion of the second jaw may provide the first and second jaws with a shape similar to a hooked-shaped beak of a bird of prey. In the longitudinal direction of the first jaw and the second jaw (e.g. measured from the pivot axle), the second jaw may have a greater length along the longitudinal direction compared to the first jaw in the closed position. The difference in length between the first jaw and the second jaw can correspond to the length of the overhang portion along the longitudinal direction. In other words, without the overhang portion, the first jaw and the second jaw may have the same length and/or configuration. The overhang portion protrudes from the second surface towards the first jaw and partially or completely covers the distal end face.

Tissue can be clamped or grasped between the distal end face and the side surface of the overhang portion. The surface areas of the side surface and the distal end face are significantly smaller than the surface areas of the first surface and the second surface. Therefore, smaller portions of tissue can be grasped between the distal end face and the side surface compared to clamping tissue between first surface and the second surface. This allows finer cutting of tissue that is clamped between the distal end face and the side surface.

According to a further aspect of the present invention, there is provided an electrosurgical apparatus for sealing and cutting tissue which comprises a generator unit for generating radiofrequency and/or microwave electromagnetic energy and the electromagnetic electrosurgical instrument as described above. The transmission line is configured to convey the radiofrequency and/or microwave electromagnetic energy from the generator unit to first electrode, the second electrode and/or the third electrode.

The generator unit may be configured to generate electromagnetic energy of a fixed single frequency or of a plurality of fixed single frequencies. Alternatively or additionally, the generator unit may be tuneable to generate electromagnetic energy of various frequencies, for example in a continuous range of frequencies between a minimum frequency and a maximum frequency. The generator unit may be connected to a power supply which provides the energy for generating the radiofrequency electromagnetic energy and/or microwave electromagnetic energy.

The generator unit is electrically and/or electronically (directly or indirectly) connected to the transmission line. Optionally, the generator unit generates the radiofrequency energy and/or microwave energy which is conveyed by the transmission line to the first to third electrodes where the radiofrequency energy and/or microwave energy is radiated into the treatment site.

In an optional embodiment, the generator unit is configured to simultaneously generate microwave electromagnetic energy of the first frequency and microwave electromagnetic energy of a second frequency.

For example, the generator unit includes a generator that is configured to simultaneously generate electromagnetic energy of two different (fixed) frequencies.

Alternatively, the generator unit includes a first generator for generating electromagnetic energy of the first frequency and a second generator for generating electromagnetic energy of the second frequency. The output of the first generator and output of the second generator can be combined using a multiplexer.

The multiplexer may be a diplexer and can combine the input from various sources into one output. For example, a multiplexer (or diplexer) is used to combine the output of first and second generators to a single output which is connected or coupled to the transmission line.

In an optional embodiment, the generator unit is configured to simultaneously or alternatingly generate microwave electromagnetic energy of the first frequency and radiofrequency electromagnetic energy of a third frequency.

The generator unit may include a first generator for generating electromagnetic energy of the first frequency, a second generator for generating electromagnetic energy of the second frequency, and/or a third generator for generating electromagnetic energy of the third frequency. The output of the first generator and the output of the second generator may be combined as described above using a multiplexer.

The output of the third generator may be combined with the output of the multiplexer using a combiner which can include a switch for alternatingly switching between outputting the output of the multiplexer and outputting the output of the third generator. The combiner may include an additional multiplexer for combining the output of the multiplexer and the output of the third generator to simultaneously emit electromagnetic energy of the first frequency, the second frequency, and the third frequency. In this case, microwave sealing and radiofrequency cutting can be simultaneously effected.

If switching between the output of microwave energy and radiofrequency energy is possible, this can be used for sealing the tissue using microwave energy and then subsequently cutting the tissue using radiofrequency energy. Alternatively, the switch between the output of microwave energy and radiofrequency energy can be executed repeatedly and rapidly providing near simultaneous cutting and sealing.

### BRIEF DESCRIPTION OF THE DRAWINGS

Embodiments of the invention are described in detail below with reference to the accompanying drawings, in which:

FIG. 1 shows a schematic view of an embodiment of an electrosurgical apparatus;

FIG. 2a shows a perspective view of an embodiment of an electrosurgical instrument (in an open position) of the electrosurgical apparatus shown in FIG. 1;

FIG. 2b shows a perspective cross-sectional view of the electrosurgical instrument (in an open position) shown in FIG. 2a;

FIG. 2c shows a perspective cross-sectional view of a first jaw of the electrosurgical instrument shown in FIG. 2a wherein a first isolating portion is removed;

FIG. 3 shows a perspective cross-sectional view of a first jaw a further embodiment of an electrosurgical instrument of the electrosurgical apparatus shown in FIG. 1 wherein a first isolating portion is removed;

FIG. 4a shows a perspective view of a further embodiment of an electrosurgical instrument (in an open position) of the electrosurgical apparatus shown in FIG. 1;

FIG. 4b shows a perspective cross-sectional view of the electrosurgical instrument (in an open position) shown in FIG. 4a;

FIG. 5a shows a perspective view of a further embodiment of an electrosurgical instrument (in an open position) of the electrosurgical apparatus shown in FIG. 1;

FIG. 5b shows a perspective view of FIG. 5a wherein a second isolating portion is removed;

FIG. 5c shows a perspective cross-sectional view of the electrosurgical instrument (in an open position) shown in FIG. 5b (i.e. the second isolating portion is removed);

FIG. 6a shows a perspective view of a further embodiment of an electrosurgical instrument (in an open position) of the electrosurgical apparatus shown in FIG. 1;

FIG. 6b shows a perspective view of the electrosurgical instrument of FIG. 6a in a closed position;

FIG. 6c shows a perspective cross-sectional view of the electrosurgical instrument (in the closed position) shown in FIG. 6b;

FIG. 7a shows a perspective view of a further embodiment of an electrosurgical instrument (in an open position) of the electrosurgical apparatus shown in FIG. 1;

FIG. 7b shows a perspective view of the electrosurgical instrument of FIG. 7a in a closed position;

FIG. 7c shows a perspective cross-sectional view of the electrosurgical instrument (in the closed position) shown in FIG. 7b;

FIG. 7d shows a perspective cross-sectional view of the electrosurgical instrument (in the open position) shown in FIG. 7a wherein a first isolating portion and a second isolating portion are removed;

FIG. 8a shows a perspective view of a further embodiment of an electrosurgical instrument (in an open position) of the electrosurgical apparatus shown in FIG. 1;

FIG. 8b shows a perspective cross-sectional view of a first jaw of the electrosurgical instrument shown in FIG. 8a;

FIG. 8c shows a cross-sectional view of the first jaw of the electrosurgical instrument shown in FIG. 8b indicating a microwave energy emission distribution at the first jaw;

FIG. 9 shows a cross-sectional view of an embodiment of an electrosurgical instrument (in a closed position) of the electrosurgical apparatus shown in FIG. 1;

FIG. 10 shows a schematic view of a further embodiment of an electrosurgical apparatus;

FIG. 11 shows a perspective cross-sectional view of a further embodiment of an electrosurgical instrument (in an open position) of the electrosurgical apparatus shown in FIG. 10;

FIG. 12a shows a perspective view of a first jaw of a further embodiment of an electrosurgical instrument (in an open position) of the electrosurgical apparatus shown in FIG. 10; and

FIG. 12b shows a perspective cross-sectional view of the first jaw of the electrosurgical instrument shown in FIG. 12a.

### DETAILED DESCRIPTION; FURTHER OPTIONS AND PREFERENCES

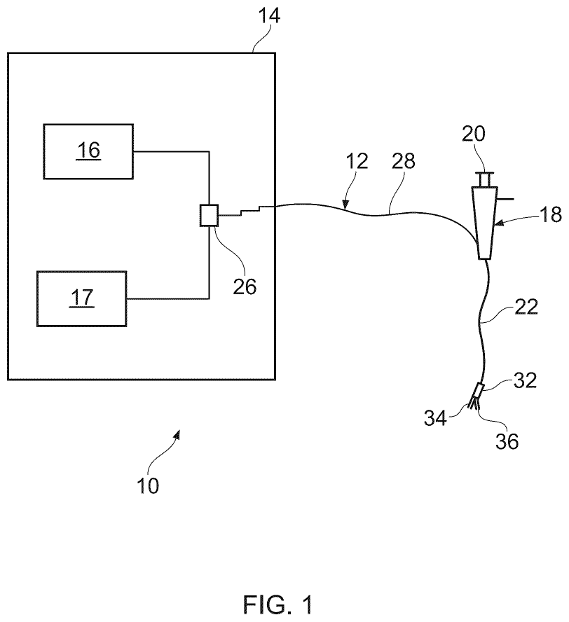

The present invention relates to an electrosurgical instrument and apparatus capable of delivering microwave energy to seal tissues (e.g. blood vessels) and of cutting the tissue. The electrosurgical instrument and apparatus may be used in open surgery but may find particular use in procedures where there is restricted access to the treatment site. For example, the electrosurgical instrument of the invention may be adapted to fit within the instrument channel of a surgical scoping device i.e. laparoscope, endoscope, or the like. FIG. 1 shows a schematic view of an electrosurgical apparatus 10 in which the electrosurgical instrument of the invention may be used.

FIG. 1 is a schematic diagram of a complete electrosurgical apparatus 10 that is an embodiment of the invention. The electrosurgical apparatus 10 is arranged to treat biological tissue using radiofrequency (RF) and/or microwave electromagnetic (EM) energy delivered from an electrosurgical instrument 12. The electromagnetic energy emitted by the electrosurgical instrument 12 into a treatment site can be used to coagulate, cut, and/or ablate tissue in the treatment site.

The electrosurgical apparatus 10 further comprises a generator unit 14 which can controllably supply radiofrequency and/or microwave electromagnetic energy to the electrosurgical instrument 12. The generator unit 14 may include a first generator 16 and a second generator 17. Suitable generators for this purpose are described in WO 2012/076844, which is incorporated herein by reference. The generator unit 14 may be arranged to monitor reflected signals received back from the electrosurgical instrument 12 in order to determine an appropriate power level for delivery. For example, the generator unit 14 may be arranged to calculate an impedance seen at the electrosurgical instrument 12 in order to determine an optimal delivery power level.

The electrosurgical apparatus 10 further comprises a surgical scoping device 18, such as a bronchoscope, endoscope, gastroscope, laparoscope or the like. The scoping device 18 may include a handpiece 20 and a flexible shaft 22. The handpiece 20 may include means for guiding the flexible shaft 22 through a cavity of a body. For example, the handpiece 20 can include means for moving a distal end of the flexible shaft 22 to change direction of the distal end of the flexible shaft 22. This helps manoeuvring the flexible shaft 22 through the cavity of the body. The flexible shaft 22 may include a working channel through which elongated structures can be moved and, thus, positioned at the treatment site within the cavity of the body.

The first generator 16 and the second generator 17 are each configured to generate electromagnetic energy of a fixed frequency. However, the generator unit 14 is not limited thereto; the first generator 16 and/or the second generator 17 can be configured to generate AC electromagnetic energy in a continuous range between a minimum frequency and a maximum frequency. The frequency of the electromagnetic energy to be generated by the first generator 16 and/or the second generator 17 may be selected using an interface (not shown in the figures).

The generator unit 14 can include a combiner 26 which is configured to temporally switch between outputting the output of the first generator 16 or the output of the second generator 17. The combiner 26 may also be configured to combine the outputs of the first generator 16 and of the second generator 17. In this case, the combiner 26 acts as a multiplexer or diplexer.

The generator unit 14 is thus capable of generating and controlling power to be delivered to the electrosurgical instrument 12, e.g. via a transmission line, which extends from the generator unit 14 through the surgical scoping device 18 and instrument channel to the distal tip of the instrument channel. The generator unit 14 may have a user interface for selecting and/or controlling the power delivered to the electrosurgical instrument 12, e.g. controlling the first and/or the second generators 16, 17 and/or the combiner 26. The generator unit 14 may have a display for showing the selected energy delivery mode. In some examples, the generator unit 14 may allow for an energy delivery mode to be selected based on the size of the vessel to be sealed.

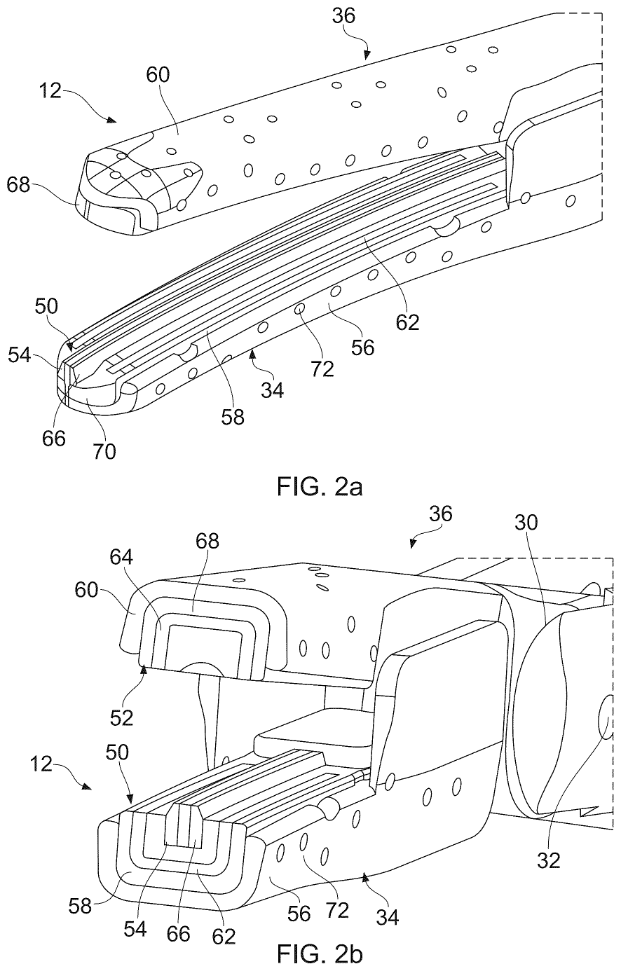

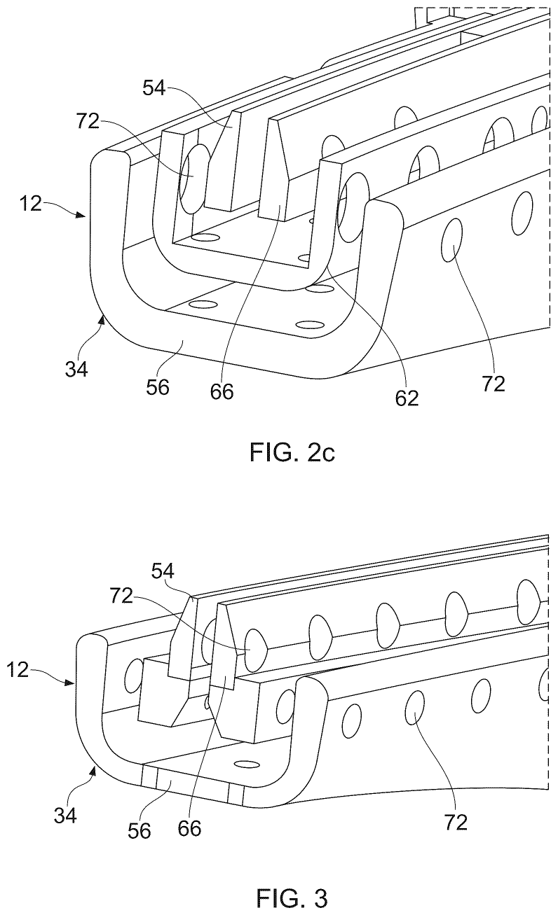

As exemplarily shown in FIGS. 2a to 2c, the electrosurgical instrument 12 can include the transmission line 28 (see FIG. 1), an instrument shaft 30, a joint 32, a first jaw 34, and/or a second jaw 36. The transmission line 28 may include a single coaxial cable that connects the generator unit 14 to the first jaw 34 and/or second jaw 34 for conveying the radiofrequency and/or microwave energy.

FIGS. 2a to 2c show schematic perspectives views and/or cross-sectional views of a distal end of an embodiment of the electrosurgical instrument 12. The second jaw 36 is rotatably or pivotally connected or coupled—via the joint 32—to the instrument shaft 30 which is dimensioned to fit within the instrument channel of the surgical scoping device 18. The first jaw 24 is rotatably or pivotally connected or coupled to the instrument shaft 30 via the joint 32. The instrument shaft 30 comprises a tubular sheath that covers the transmission line for carrying microwave and/or radiofrequency energy to the jaws 34, 36 together with various control wires or (actuation) rods that are arranged to control and/or physically manipulate the first and second jaws 34, 36, as discussed below.

The first jaw 34 and the second jaw 36 are operably coupled to the joint 32 that is mounted on a distal end of the instrument shaft 30. So, the pair of jaws 34, 36 are both pivotable or rotatable. The joint 32 may be arranged to ensure that the jaws remain laterally aligned as they are moved together.

In an alternative embodiment, one of the first and second jaws 34, 36 may be arranged not pivot relative to the joint 32, e.g. is fixedly attached to the shaft 30.

In the embodiment shown, the joint 32 includes a pivot axle (not visible in FIGS. 2a to 2c) which defines a pivot axis. The first jaw and the second jaw 36 can pivot around the pivot axis or pivot axle. For example, the pivot axle is fixed to the joint 32 and the first jaw 32 and the second jaw 36 can rotate around the pivot axle.

The joint 32 may have a clevis structure. The first jaw 34 and/or the second jaw 36 may include elongated slots. A control wire or actuation rod can include a cam which is inserted in the slots of the first jaw 34 and/or the second jaw 36. The engagement of the cam with the slots provides an actuation mechanism which translates a back-and-forth movement of the control wire (and thus of the cam) into a rotational movement of the first jaw 34 and the second jaw 36 around the pivot axle.

In use, the first jaw 34 and the second jaw 36 are intended to grip biological tissue (in particular a blood vessel) therebetween. The first jaw 34 and the second jaw 36 are arranged to apply pressure to the biological tissue between the opposed surfaces of the jaws 34, 36 and deliver energy (preferably microwave and/or radiofrequency electromagnetic energy) into the tissue from the transmission line.

The first jaw 34 includes a first surface 50 which opposes a second surface 52 of the second jaw 36. The first surface 50 and/or the second surface 52 may form an outer surface of the first jaw 34 and the second jaw 36 respectively, which can be brought into contact with each other when the jaws 34, 36 are in the closed position. For example, the first surface 50 and the second surface 52 may be considered pressure pads or pressure areas with which pressure can be applied to the tissue grasped between the first jaw 34 and the second jaw 36.

In the embodiment of FIGS. 2a to 2c, the first jaw 34 and the second jaw include a first electrode 54, a second electrode 56, a first isolating portion 58, a third electrode 60, a fourth electrode 62, fifth electrode 64, a sixth electrode 66, and/or a second isolating portion 68. The first electrode 54, the second electrode 56, the third electrode 60, the fourth electrode 62, the fifth electrode 64, and/or the sixth electrode 66 are made from an electrically conductive material, such as metal or a metal alloy. The second electrode 56 may form the outer surface of the first jaw 34 and/or may provide the connection to the instrument shaft 30. Thus, the second electrode 56 may have a function of providing the stability of the first jaw 34.

The second electrode 56 may have a form of a half-shell in a portion along the first surface 50. The second electrode 56 may surround the first electrode 54, the first isolating portion 58, the fourth electrode 62, and/or the sixth electrode 66. This means that the first electrode 54, the first isolating portion 58, the fourth electrode 62, and/or the sixth electrode 66 may be embedded in the half-shell of the second electrode 56. The shape of the second electrode 56 may also be considered as providing a recess or channel in which the first electrode 54, the first isolating portion 58, the fourth electrode 62, and/or the sixth electrode 66 are arranged.

The first isolating portion 58 electrically isolates the first electrode 54, the second electrode 56, the fourth electrode 62, and/or the sixth electrode 66 from each other. The first isolating portion 58 may be made from an electrically non-conductive material such as ceramics (e.g. including Zirconia), PEEK, silicone, and/or other plastic materials.

Preferably, the first isolating portion 58 is made from an elastically deformable material, such as silicone. Further, the first electrode 54, the fourth electrode 62, and/or the sixth electrode 66 may be floating. This means that the first electrode 54, the fourth electrode 62, and/or the sixth electrode 66 are only attached to and supported by the first isolating portion 58. Due the elastic properties of the first isolating portion 58, the first electrode 54, the fourth electrode 62, and/or the sixth electrode 66 can move relative to each other and/or relative to the second electrode 56 when a pressure is applied, e.g. a tissue is grasped and squeezed between the first jaw 34 and the second jaw 36. The first electrode 54, the fourth electrode 62, and the sixth electrode 66 may be flexibly connected to the transmission line 28 for allowing movement relative to the second electrode 58 and, thus, the transmission line.

The second electrode 56 may have a U-shape or V-shape in a cross-sectional view along a section of the first jaw 34. Further, the second electrode 56 may have a plate-shape (along this section of the first jaw 34). At a distal end and/or a proximal end of the first jaw 34, the second electrode 56 may have a different configuration so that the second electrode 56 does not have an open end but a closed end (not shown in the figures). This means that the second electrode 56 can shield the first electrode 54, the fourth electrode 62, and/or the sixth electrode 66 in all directions away from the first surface 50.

The first electrode 54, the second electrode 56, the fourth electrode 62, and/or the sixth electrode 66 are exposed at the first surface 50 for getting in contact with the tissue clamped between the first jaw 34 and the second jaw 36. However, the second electrode 56 may be offset and not flush with the first surface 50. Upon compression of the first isolating portion 58, the second electrode 56 may get in contact with the tissue.

Two sections of the second electrode 56 and two sections of the fourth electrode 62 are exposed on the first surface 50 which are spaced from each other. In the embodiment shown, the two exposed sections are straight and extend in a direction of extension of the first jaw 34.

The first electrode 54 and the sixth electrode 66 are configured to emit radiofrequency energy for tissue cutting. The first electrode 54 is electrically isolated from the sixth electrode 66. The first electrode 54 may be an active electrode for delivering radiofrequency electromagnetic energy while the sixth electrode 66 is a counter electrode for providing bipolar radiofrequency cutting. The first electrode 54 and the sixth electrode 66 are connected to the transmission line 28, for example to respective wires of the transmission line 28.

The second electrode 56 and the fourth electrode 62 are configured to emit microwave energy for tissue sealing. The second electrode 56 is electrically isolated from the fourth electrode 62. The fourth electrode 62 may be an active electrode for delivering microwave electromagnetic energy while the second electrode 56 is a return electrode. The fourth electrode 62 is connected to the transmission line 28, optionally an inner conductor the coaxial cable. The second electrode 56 is connected to the transmission line 28, optionally an outer conductor the coaxial cable.

The first electrode 54 and the sixth electrode 66 are exposed on the first isolating portion 58 and/or protrude from the first isolating portion 58. The first electrode 54 and the sixth electrode 66 are arranged to first contact tissue that is clamped between the first jaw 34 and the second jaw 36. In other words, the first electrode 54 and the sixth electrode 66 protrude from the first isolating portion 58 in a direction towards the second jaw 36. The first electrode 54 and/or the sixth electrode 66 may be a ridge or bar made from an electrically conductive material, such a metal or a metal alloy.

The fourth electrode 62 may also be exposed on the on the first isolating portion 58 but does not protrude from the first isolating portion 58. Rather, the fourth electrode 62 may also be flush with the first isolating portion 58 on the first surface 50. The fourth electrode 62 may have a U-shape or V-shape in a cross-sectional view along a section of the first jaw 34. Further, the fourth electrode 62 may have a plate-shape (along this section of the first jaw 34). End faces of the plate-shaped fourth electrode 62 may constitute the exposed sections of the fourth electrode 62. At a distal of the first jaw 34, the fourth electrode 62 may not be exposed, but completely embedded or buried within the first isolating portion 58.

The first electrode 54 and the sixth electrode 66 are arranged in the middle between the respective two sections of the second electrode 56 and the fourth electrode 62 that are exposed on the first surface 50. The exposed sections of the second electrode 56, of the fourth electrode 62, and of the first isolating portion 58 may be symmetrical to the exposed sections of the first electrode 54 and the sixth electrode 66. The first electrode 54 and the sixth electrode 66 may divide the first surface 50 into a first face and a second face. The first face and the second face may be symmetrical to the first electrode 54 and the sixth electrode 66. The first face as well as the second face may each include an exposed section of the second electrode 56, an exposed section of the fourth electrode 62, and/or an exposed section of the first isolating portion 58.

The first face and the second face define an angle of 180° with respect to each other. As the first electrode 54 and the sixth electrode 66 are arranged between the first face and the second face, the first electrode 54 and the sixth electrode 66 protrude from both the first face and the second face.

The first jaw 34 further includes a distal end face 70 which is directly adjacent to the first surface 50. The distal end face 70 is inclined to the first surface 50 by an angle of 90°.

The distal end face 70 is a side surface of the first jaw 34 that firstly comes in contact with tissue if the first jaw 34 is advanced in the longitudinal direction of the first jaw 34 towards the tissue. In the embodiment of FIGS. 2a to 2c, the distal end face 70 is curved. The first electrode 54, the second electrode 56, the sixth electrode 66, and/or the first isolating portion 58 are exposed on the distal end face 70. The sections of the first electrode 54, the second electrode 56, and/or the sixth electrode 66 that are exposed on the distal end face 70 are spaced from each other. Further, the sections of the first electrode 54 and the sixth electrode 66 that are exposed on the distal end face 70 are arranged between the sections of the second electrode 56 that are exposed on the distal end face 70. The sections of the first electrode 54, the second electrode 56, and/or the sixth electrode 66 that are exposed on the distal end face 70 are continuous with the respective sections of the first electrode 54, the second electrode 56, and the sixth electrode 66 that exposed on the first surface 50. As such, the sections of the first electrode 54 and the sixth electrode 66 that exposed on the distal end face 70 provide one active electrode and a counter electrode for radiofrequency cutting.

The surface area of the distal end face 70 is significantly smaller than the surface area of the first surface 50. Similarly, the sections of the first electrode 54 and the sixth electrode 66 that are exposed on the distal end face 70 are smaller than the respective sections exposed on the first surface 50. This allows fine cutting using the exposed sections of the first electrode 54 and sixth electrode 66 on the distal end face 70. For example, the exposed sections of the first electrode 54 and sixth electrode 66 on a distal end face 70 may be used to cut holes into the tissue.

The second jaw 36 includes the third electrode 60, the fifth electrode 64, and/or the second isolating portion 68. The first electrode 54 and the sixth electrode 66 protrude from the first isolating portion 58 towards the second jaw 36 and contact the second isolating portion 68 in the closed position. The second isolating portion 68 may include an elongate recess extending in the longitudinal direction of the second jaw 36 for receiving the protruding portions of the first electrode 54 and the sixth electrode 66.

The third electrode 60 and/or the fifth electrode 64 are made from an electrically conductive material, such as metal. The third electrode 60 may form the outer surface of the second jaw 36 and/or may provide the connection to the pivot axle. Further, the slots of the second jaw 36 may be arranged on the third electrode 66. Thus, the third electrode 66 may have a function of providing the stability of the second jaw 36.

The third electrode 60 may have a form of a half-shell in a portion of the second surface 52. In the embodiments of FIGS. 2a to 2c, the third electrode 60 may surround the fifth electrode 64 and the second isolating portion 68. This means that the fifth electrode 64 and the second isolating portion 68 may be embedded in the half-shell of the third electrode 60. The shape of the third electrode 60 may also be considered as providing a recess or channel in which the fifth electrode 64 and the second isolating portion 68 are arranged.

The third electrode 60 may be mirror-symmetric to the second electrode 56. The third electrode 60 may have a U-shape (see FIG. 2b) in a cross-sectional view along a section of the second jaw 36. Further, the third electrode 60 may have a plate-shape (see FIGS. 2a to 2c) along this section of the second jaw 36. At a distal end of the second jaw 36, the third electrode 60 has the same configuration so that the third electrode 60 has an open end but a not a closed end. This means that the third electrode 60 can shield the first electrode 54 and the sixth electrode 66 in all directions away from the second surface 52. The second electrode 56 and the third electrode 60 may be electrically connected to each other, either directly or by being connected to the same conductor of the transmission line.

The fifth electrode 64 may have a U-shape or V-shape in a cross-sectional view along a section of the second jaw 36. Further, the fifth electrode 64 may have a plate-shape (along this section of the second jaw 36). The fifth electrode 64 is exposed on the second surface 52. End faces of the plate-shaped fifth electrode 64 may constitute the exposed sections of the fifth electrode 64. At a distal of the second jaw 36 (e.g. at a distal end face 70 thereof), the fifth electrode 64 may not be exposed, but completely embedded or buried within the second isolating portion 68.

The first electrode 54, the fourth electrode 62, the fifth electrode 64, and the sixth electrode 66 each include through-holes 72 which are filled by the first isolating portion 58 and the second isolating portion 68, respectively. So, the first electrode 54, the fourth electrode 62, the fifth electrode 64, and the sixth electrode 66 interlock with the first isolating portion 58 and the second isolating portion 68, respectively. As such, the first electrode 54, the fourth electrode 62, the fifth electrode 64, and the sixth electrode 66 are attached to the first isolating portion 58 and the second isolating portion 68, respectively. The positive fit of the first electrode 54, the fourth electrode 62, the fifth electrode 64, and the sixth electrode 66 with the first isolating portion 58 and the second isolating portion 68, respectively, is helpful if the first isolating portion 58 and the second isolating portion 68 are made from a non-adhesive or non-stick material, such as silicone.

Similarly, the second electrode 56 and the third electrode 60 include through-holes 72 in which portions of the first isolating portion 58 and the second isolating portion 68, respectively, are arranged. So, the first isolating portion 58 and the second isolating portion 68 interlock with the second electrode 56 and the third electrode 60, respectively. In other words, the first isolating portion 58 and the second isolating portion 68 are attached to the second electrode 56 and the third electrode 60, respectively, by form fit. Further, the first electrode 54, the fourth electrode 62, and/or the sixth electrode 66 are attached to the second electrode 56 via and (solely) by the first isolating portion 58. Further, the fifth electrode 64 is attached to the third electrode 60 via and (solely) by the second isolating portion 68.

The embodiment of the electrosurgical instrument 12 shown in FIG. 3 includes the same features, characteristics, and/or optional embodiment as the embodiment of the electrosurgical instrument 12 shown in FIGS. 2a to 2c except for the following differences.

The electrosurgical instrument 12 of FIG. 3 does not include the fourth electrode 62 and the fifth electrode 64. The first electrode 54 and the sixth electrode 66 additionally act as an active electrode for microwave sealing. The second electrode 56 as well as the third electrode 60 may act as return electrodes for the microwave sealing. This means, that the first electrode 54 and the sixth electrode 66 have a double functionality of providing radiofrequency cutting therebetween and microwave sealing with the second electrode 56 and the third electrode 60.

The first electrode 54 and the sixth electrode 66 are again floating, e.g. are movably supported by and attached to the first isolating portion 58. The second isolating portion 68 may not support any electrode and may provide an abutting surface against which the first electrode 54 and the sixth electrode 66 abuts in a closed position of the first jaw 34 and the second jaw 36.

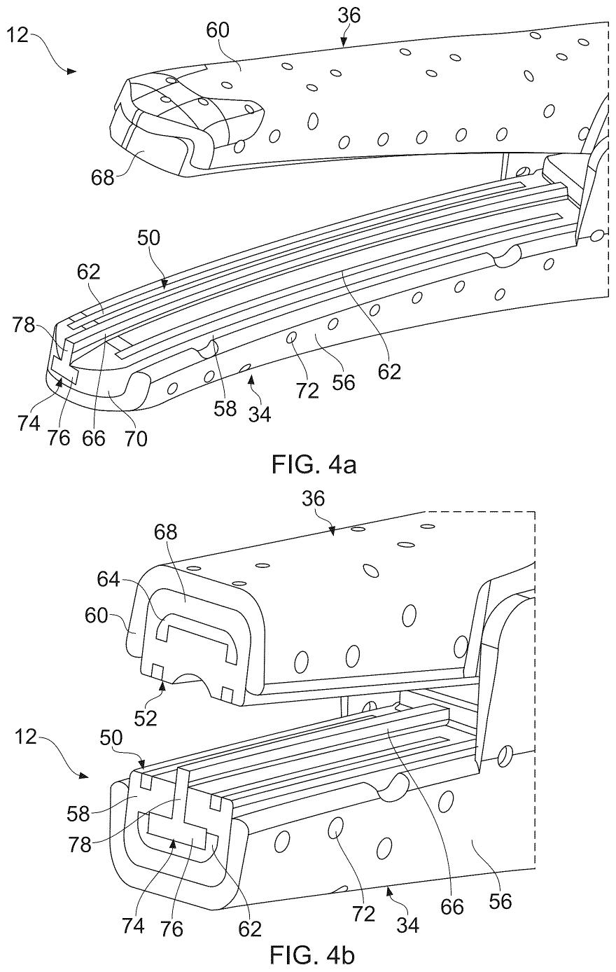

The embodiment of the electrosurgical instrument 12 shown in FIGS. 4a and 4b includes the same features, characteristics, and/or optional embodiment as the embodiment of the electrosurgical instrument 12 shown in FIGS. 2a to 2c except for the following differences.

The first electrode 54 and the sixth electrode 66 are metallic strips that are attached to an electrode support 74 which can be made from an electrically insulating material such as ceramic or alumina (Aluminium oxide Al2O3). The electrode support 74 may have a T-shaped in a cross-sectional view of the first jaw 34. The electrode support 74 may include a support base plate 76 and a support rib 78 which are fixedly attached to each other or are a unitary component. The support base plate 76 interlocks with the first isolating portion 58 so that the electrode support 74 is attached to and supported by the first isolating portion 58. So, the support base plate 76 provides a form fit for the support rib 78. The first electrode 54 (not shown in FIGS. 4a and 4b) and the sixth electrode 66 are attached on opposing side surfaces of the support rib 78 for providing to spatially separated electrodes for radiofrequency cutting.

The support base 76 plate may be in direct contact with a bottom surface of the fourth electrode 62. This is possible since the support base plate 76 is made from an electrically insulating material.

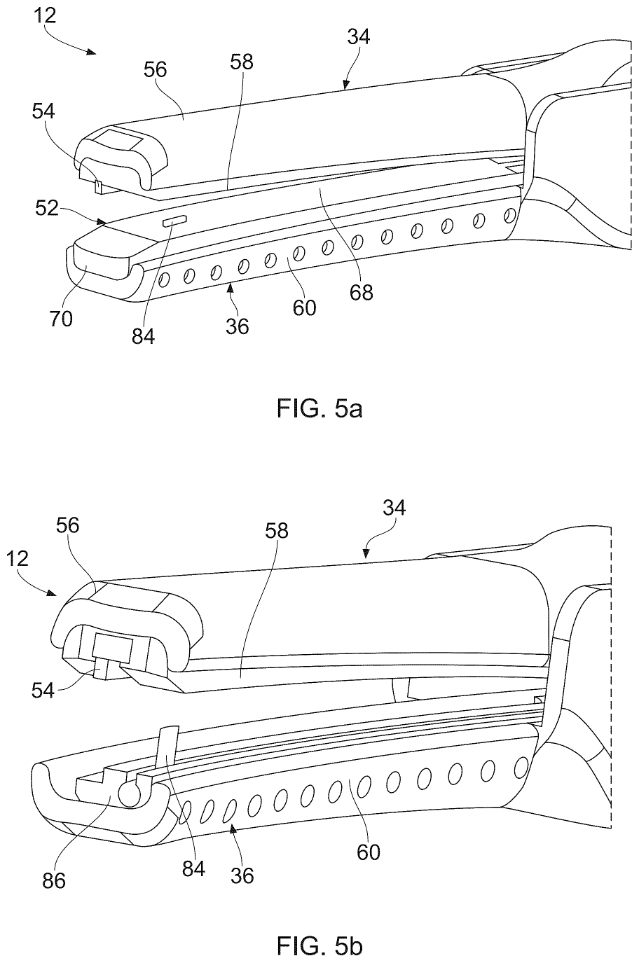

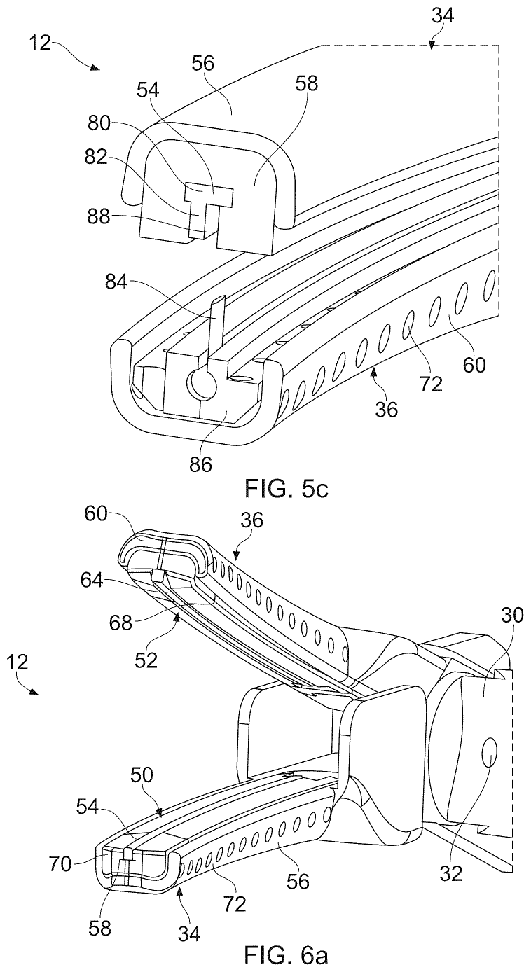

The embodiment of the electrosurgical instrument 12 shown in FIGS. 5a to 5c includes the same features, characteristics, and/or optional embodiment as the embodiment of the electrosurgical instrument 12 shown in FIGS. 2a to 2c except for the following differences.

The electrosurgical instrument 12 does not include the fourth electrode 62, the fifth electrode 64, and the sixth electrode 66. In FIGS. 5a to 5c, the first jaw 34 is the upper jaw and the second jaw 36 is the lower jaw.

The first electrode 54 does not include through-holes 72 for providing a form fit with the first isolating portion 58. Instead, the first electrode 54 may have a T-shaped in a cross-sectional view of the first jaw 34 (see FIG. 5c). The first electrode 54 may include a base plate 80 and a rib 82 which are fixedly attached to each other (e.g. by welding) or are a unitary component. The base plate 80 and/or the rib 82 can be made from an electrically conductive material, such as metal or a metal alloy.

The base plate 80 interlocks with the first isolating portion 58 so that first electrode 54 is attached to and supported by the first isolating portion 58. So, the support base 80 provides a form fit for the rib 82. The first electrode 54 is again floating, e.g. can move relative to the second electrode 56 when pressure is applied to the first electrode 54. The rib 82 is exposed on the first surface 50 but does not protrude from the first surface 50. For example, in the closed position, the first isolating portion 58 is compressed so that the first electrode 54 contacts tissue grasped between the first jaw 34 and the second jaw 36.

The first electrode 54 may act as an active electrode for microwave sealing for which the second electrode 56 and the third electrode 60 can act as ground electrodes. Further, the first electrode 54 may be an active electrode for radiofrequency cutting for which the second electrode 56 and the third electrode 60 can act as return electrodes. So, in an optional embodiment of the electrosurgical instrument 12 shown FIGS. 5a to 5c, the first, second, and third electrodes 54, 56, and 60 each provide microwave sealing and radiofrequency cutting.

The electrosurgical instrument 12 may further include a blade 84 slidable along a guide rail 86. The second isolating portion 68 includes a slot or groove along with the blade 84 is movable. The blade 84 may protrude from the second surface 52/or from the slot or groove within the second isolating portion 68. The movement of the blade 84 is defined by the guide rail 86 which includes a channel along which the blade 84 can be moved, for example using an actuation rod or the like. The guide rail 86 may be made from an electrically isolating material, e.g. ceramic or alumina (Aluminium oxide Al2O3). The guide rail 86 may be attached to the third electrode 60 and may extend in the channel provided by the third electrode 60. The guide rail 86 may provide an electrical isolation between the blade 84 and the third electrode 60.

The blade 84 may comprise a rigid element with a sharp edge adapted to slice biological tissue, e.g. a scalpel-type blade or the like. This type of blade 84 is configured to perform a “cold” cut. In this case, the first electrode 54 may not be configured for radiofrequency cutting and may only be act as an active electrode for microwave sealing.

The blade 84 may protrude into a gap 88 which is provided between a side surface of the rib 82 and the first isolating portion 58. For example, a portion of the base plate 80 is exposed when other portions of the base plate 80 are covered by the first isolating portion 58 for providing an undercut so that the first electrode 54 interlocks with the first isolating portion 58. The gap 88 may provide a channel or recess for receiving the blade 84 in the closed position.

In an optional embodiment of the electrosurgical instrument 12 shown FIGS. 5a to 5c, the blade 84 may be spatially offset to and not in contact with the rib 82 and the base plate 80. So, even in the closed position of the first jaw 34 and the second jaw 36, the blade 84 does not contact the first electrode 54. Thus, the blade 84 may provide a return electrode for the first electrode 54 acting as an active electrode for radiofrequency cutting between the blade 84 and the first electrode 54. So, in this embodiment, the blade 84 not only provides a cold cut but contributes to radiofrequency cutting.

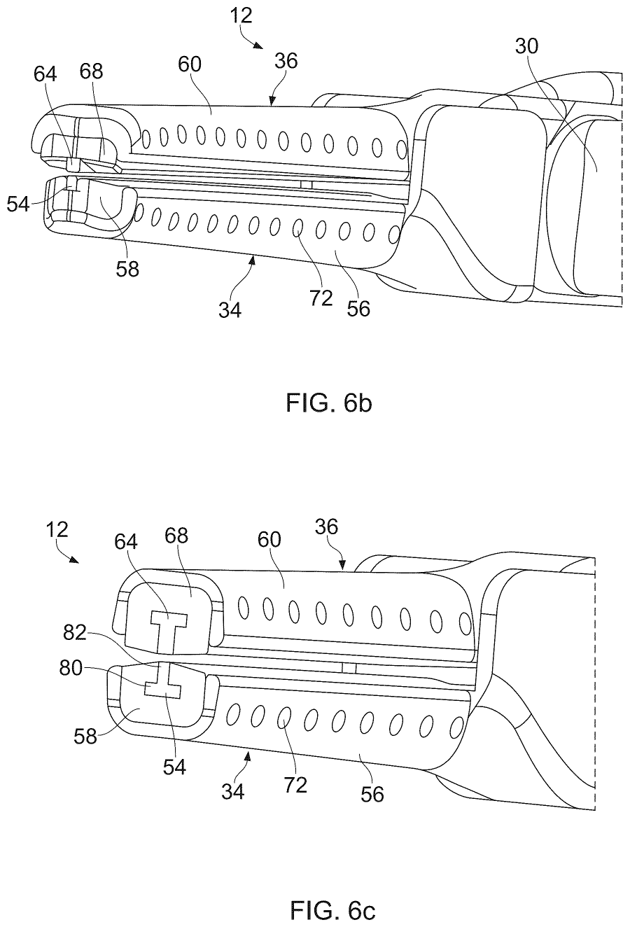

The embodiment of the electrosurgical instrument 12 shown in FIGS. 6a to 6c includes the same features, characteristics, and/or optional embodiment as the embodiment of the electrosurgical instrument 12 shown in FIGS. 2a to 2c except for the following differences.

The electrosurgical instrument 12 does not include the fourth electrode 62 and the sixth electrode 66. The first electrode 54 and the fifth electrode 64 each have a T-shape in the cross-sectional view of the first jaw 34 and the second jaw 36, respectively. The fifth electrode 64 may have the same characteristics, optional features, and optional embodiments as the T-shaped first electrode 54 described in connection with FIGS. 5a to 5c. The fifth electrode 64 may be mirror-symmetric to the first electrode 54 in the closed position of the first jaw and the second jaw 36. The base plate 80 of the first electrode 54 and/or the fifth electrode 64 may be completely buried in the first isolating portion 58 and/or the second isolating portion 68, respectively. The gap 88 may not be present.

The first electrode 54 and the fifth electrode 64 may be exposed on the first surface 50 and the second surface 52, respectively. The first electrode 54 and the fifth electrode 64 may contact each other in the closed position, for example along their entire length over which the first electrode 54 and the fifth electrode 64 are exposed on the respective surfaces 50, 52. In the closed position, the first electrode 54 and the fifth electrode 64 may be the only components of the first jaw 34 and the second jaw 36 that are in contact with each other. Upon the application of pressure on the tissue grasped between the first jaw 34 and the second jaw 36, the first electrode 54 and the fifth electrode 64 may move away from each other so that the first electrode 54 and the fifth electrode 64 are compressed into the first isolating portion 58 and the second isolating portion 68, respectively.

The first electrode 54 may be an active electrode for radiofrequency cutting and the fifth electrode 64 may be a return electrode for radiofrequency cutting. So, in the embodiment shown in FIGS. 6a to 6c, the radiofrequency cutting is provided between the first jaw 34 and the second jaw 36 which is different to the other embodiments shown in the figures where the radiofrequency cutting is provided along the first jaw 34 or the second jaw 36. So, the currents flowing during radiofrequency cutting flow through the tissue in the in the embodiment shown in FIGS. 6a to 6c.

The first electrode 54 and the fifth electrode 64 may each be active electrodes for microwave sealing, e.g. the first electrode 54 and the fifth electrode 64 may be both connected to the same conductor for microwave sealing, such as the inner conductor of the coaxial cable. The which the second electrode 56 and the third electrode 60 can act as return electrodes can be ground electrodes for microwave sealing.

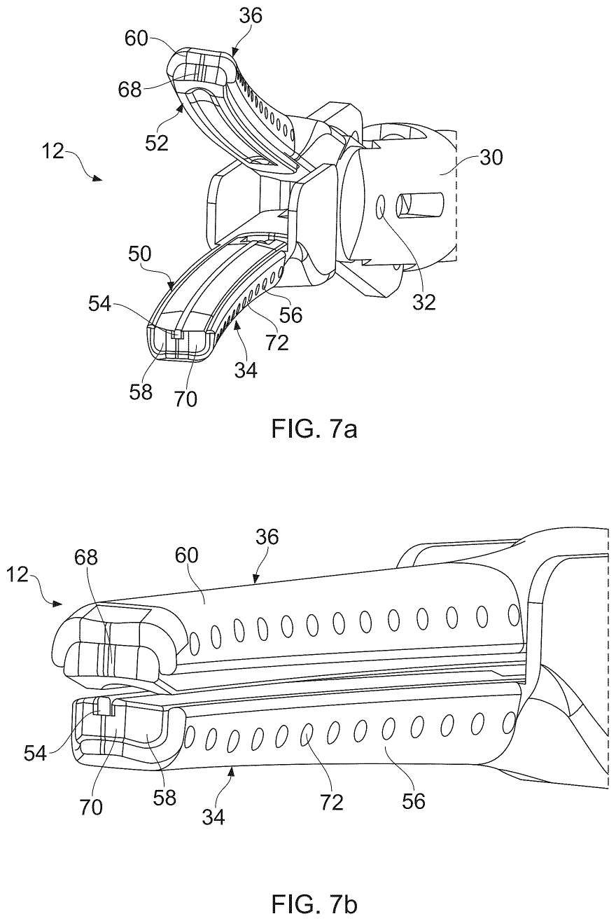

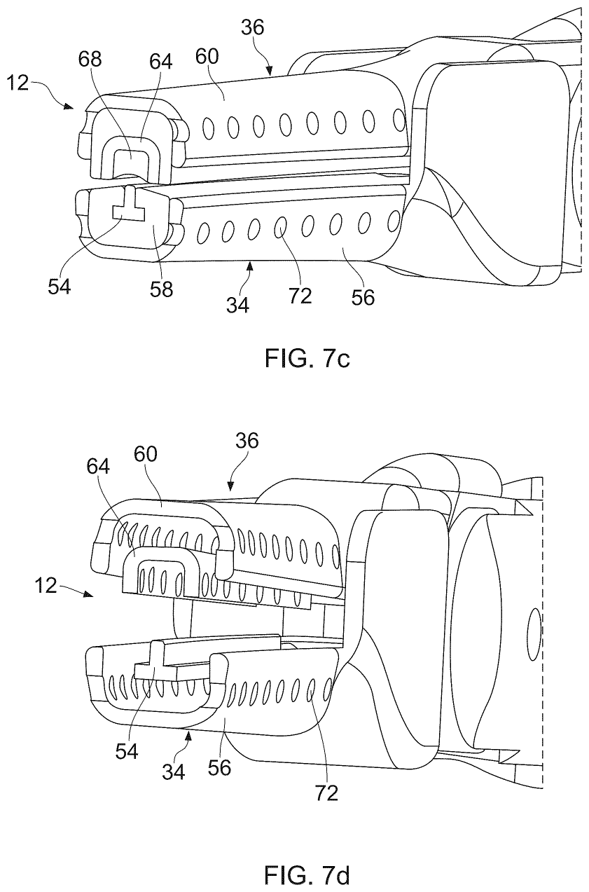

The embodiment of the electrosurgical instrument 12 shown in FIGS. 7a to 7d includes the same features, characteristics, and/or optional embodiment as the embodiment of the electrosurgical instrument 12 shown in FIGS. 2a and 2b except for the following differences.

The electrosurgical instrument 12 does not include the fourth electrode 62 and the sixth electrode 66. The fifth electrode 64 may have a U-shape in a cross-sectional view along the second jaw 36, e.g. similar to the embodiment shown in FIGS. 2a to 2c but different to the embodiment shown in FIGS. 6a to 6c. The fifth electrode 64 may be completely embedded or buried in the second isolating portion 68. In other words, the fifth electrode 64 is not exposed at the second surface 52. This reduces the risk that the tissue sticks to the fifth electrode 64. Further, due to the non-stick properties of material of the second isolating portion 68 (e.g. silicone), the risk that the tissue sticks to the second surface 52 is reduced compared to vessel sealers of the prior art.

The first electrode 54 may have the same characteristics, optional features, and optional embodiments as the T-shaped first electrode 54 of the embodiment shown in FIGS. 6a to 6c. The first electrode 54 may be an active electrode for radiofrequency cutting and the second electrode 56 as well as the third electrode 60 may be return electrodes for radiofrequency cutting. The fifth electrode 64 may be the active electrode for micro wave sealing and the and the second electrode 56 as well as the third electrode 60 may be ground electrodes for microwave sealing. The thickness of the second isolating portion 68 over the end faces of the plate-shaped fifth electrode 64 may be small so that only a thin layer of the second isolating portion 68 covers the end faces of the plate-shaped fifth electrode 64. The thinner the second isolating portion 68 is over the end faces of the plate-shaped fifth electrode 64, the more microwave energy is emitted by the fifth electrode 64.

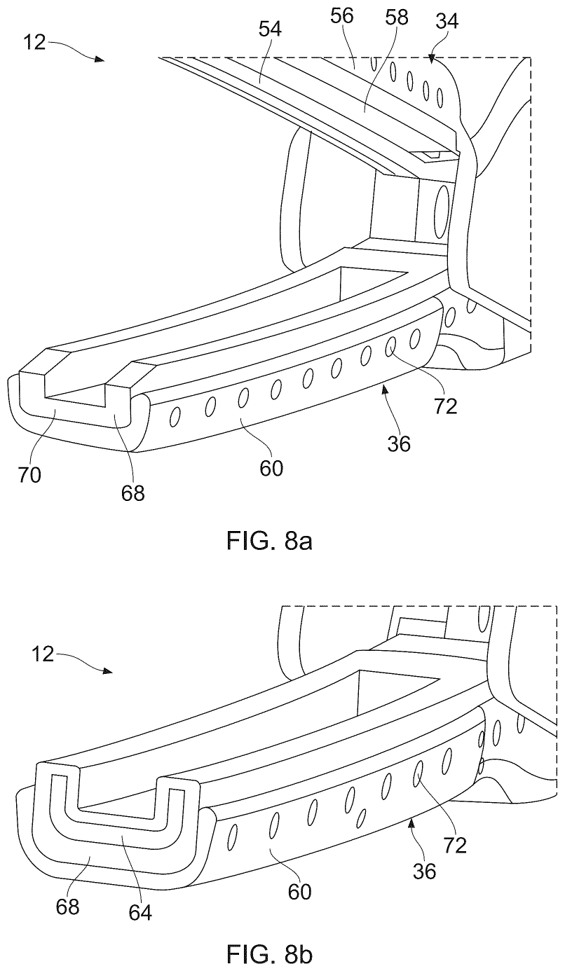

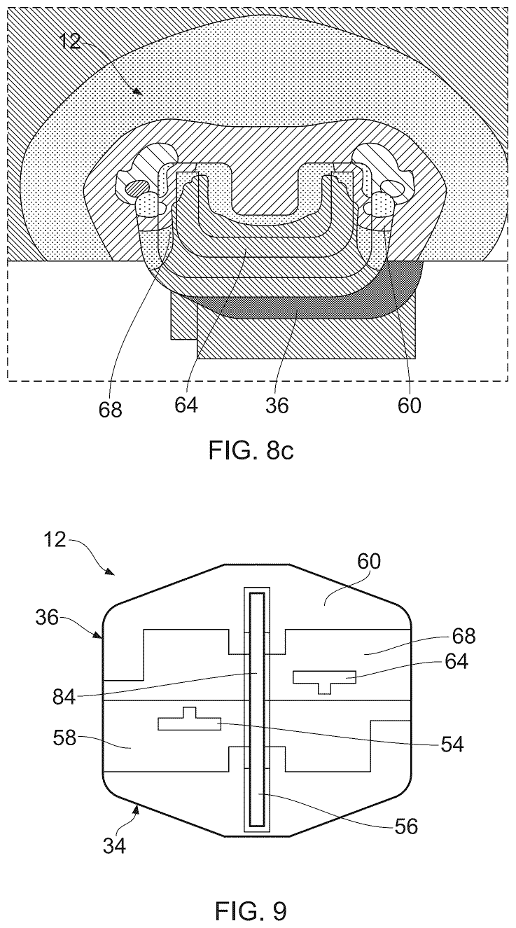

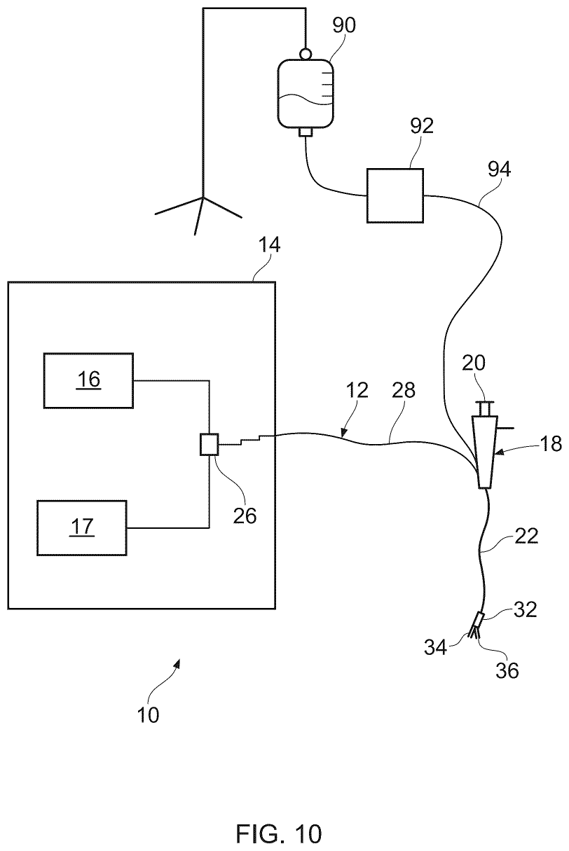

The embodiment of the electrosurgical instrument 12 shown in FIGS. 8a to 8c includes the same features, characteristics, and/or optional embodiment as the embodiment of the electrosurgical instrument 12 shown in FIGS. 7a to 7d except for the following differences.

In FIGS. 8a to 8c, the first jaw 34 is the upper jaw and the second jaw 36 is the lower jaw. The second isolating portion 68 is shaped to provide an open channel or slot for receiving the first electrode in the closed position. The second isolating portion 68 may have a similar shape as the U-shaped fifth electrode 64. In this way, the pressure on the tissue provided by the first electrode 54 can be reduced in the closed position as the tissue is pushed into the channel provided by the second isolating portion 68 by the first electrode and, therefore, is less squeezed between the first electrode 54 and the second isolating portion 58.

FIG. 8c shows the microwave emission spectrum of the fifth electrode 64 and the third electrode 60. It is apparent that the intensity of the microwave energy and, therefore, the effect of the microwave sealing is highest between the fifth electrode 64 and the third electrode 60 along the second surface 52.

The embodiment of the electrosurgical instrument 12 shown in FIG. 8 includes the same features, characteristics, and/or optional embodiment as the embodiment of the electrosurgical instrument 12 shown in FIGS. 6a to 6c except for the following differences.

The T-shaped first electrode 54 and the T-shaped fifth electrode 64 are not mirror symmetric to each other but rotationally symmetric in the closed position. The first electrode 54 and the fifth electrode 64 are spaced apart from each other along a direction perpendicular to the longitudinal direction in the closed position. So, the first electrode 54 and the fifth electrode 64 are not in contact which each other in the closed position even if they were exposed on the first surface 50 and the second surface, respectively. However, the first electrode 54 and the fifth electrode 64 are completely embedded or buried within the first isolating portion 58 and the second isolating portion 68, respectively. In other words, the first isolating portion 58 completely covers the first electrode 54 and the second isolating portion 68 completely covers the fifth electrode 64. The first electrode 54 and the fifth electrode 64 may be active electrodes for microwave sealing. An exposed section of the second electrode 56 and the third electrode 60 which is closest to the respective first electrode 54 and fifth electrode 64, respectively, may act as ground electrodes for microwave sealing.

In the embodiment shown in FIG. 9, the second electrode 56 and the third electrode 60 do not have a U-shape in a cross-sectional view as with the other embodiments shown herein. Rather, the second electrode 56 and the third electrode 60 have a L-shaped in a cross-sectional view. The second electrode 56 and the third electrode 60 may also be rotationally symmetric to each other in the closed position. So, unlike the other embodiments shown herein, the second electrode 56 and the third electrode 60 may only include one section that is exposed on or close to the first surface 50 and the second surface 52, respectively. In the embodiment shown in FIG. 9, the first isolating portion 58 completely covers the second electrode 56 and the second isolating portion 68 completely covers the third electrode 60.

The electrosurgical instrument 12 may further include the blade 84 which may be guided by slots within the second electrode 56 and the third electrode 60. So, this embodiment of the electrosurgical instrument 12 does not include a guide rail 86 that is made from an electrically isolating material. Rather, the blade 84 is in electrical contact with the second electrode 56 and the third electrode 60. The blade 84 may provide a cold cut.

The embodiment of the electrosurgical apparatus 10 shown in FIG. 1 includes the same features, characteristics, and/or optional embodiment as the embodiment of the electrosurgical apparatus 10 shown in FIG. 1 except for the following differences.

The electrosurgical apparatus 10 further includes a liquid reservoir 90 and a valve 92. The liquid reservoir 90 provides a reservoir or container for a liquid that can be supplied to the treatment site, e.g. a saline solution. The liquid reservoir may be an IV drip (a container containing liquid for intravenous (IV) therapy). The liquid reservoir 90 may be attached to a stand so that the liquid is supplied by gravitational force. The amount of liquid that is supplied to the treatment site can be controlled by the valve 92 which may include a solenoid pinch valve for opening, partially opening, or closing a pipe or hose supplying the liquid from the liquid reservoir to the valve 92.

The electrosurgical instrument 12 may further include a liquid feed 94 (e.g. including a flexible hose and/or pipe) that can be connected to the valve 92. The liquid feed 94 may protrude from the proximal end of the shaft 30 so that the liquid feed 94 can be connected to the valve 92. A distal end of the liquid feed 94 may be attached to the first jaw 32 as outlined below.

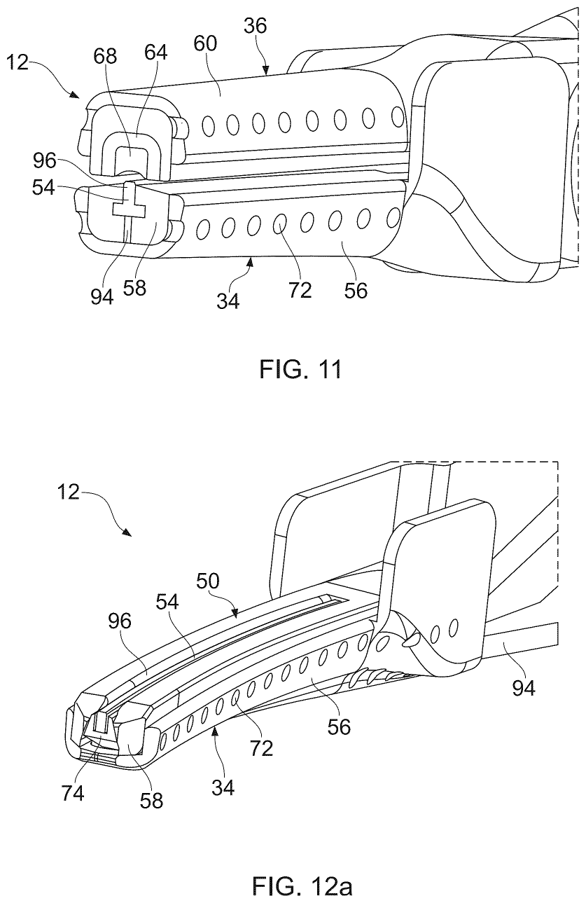

The embodiment of the electrosurgical instrument 12 shown in FIG. 11 includes the same features, characteristics, and/or optional embodiment as the embodiment of the electrosurgical instrument 12 shown in FIGS. 7a to 7d except for the following differences.

The electrosurgical instrument 12 includes the liquid feed 94 and a liquid output 96. The liquid feed 94 includes a slit in the first isolating portion 58 which extends below the first electrode 54. This slit of the fluid feed 94 is connected to the hose or pipe of the fluid feed 94 that extends in the shaft 30. So, the liquid feed 94 supplies liquid into the first jaw 34 over the (complete) length of the first electrode 54.

The liquid output 96 is—in the embodiment shown in FIG. 11—a gap between the first electrode 54 and the first isolating portion 58. Thus, the liquid can flow from the liquid feed 94, along the first electrode 54 to the first surface 50. Thus, outlets of the liquid output extend on both sides of the rib 82 of the first electrode 54. The liquid output 96 may be regarded an elongate opening along the first electrode 54. So, the liquid output 96 provides liquid over the (complete) extension of the first electrode 54. If the first electrode 54 is used for radiofrequency cutting, liquid is supplied in the around the active electrode for wetting the tissue so that the radiofrequency cut can be effected.

The gap between the first electrode 54 and the first isolating portion 58 may be so thin that the liquid is sucked by capillary action from the liquid output 94 to the first surface. As such, the gap can provide a constant supply of liquid at the first surface 50.

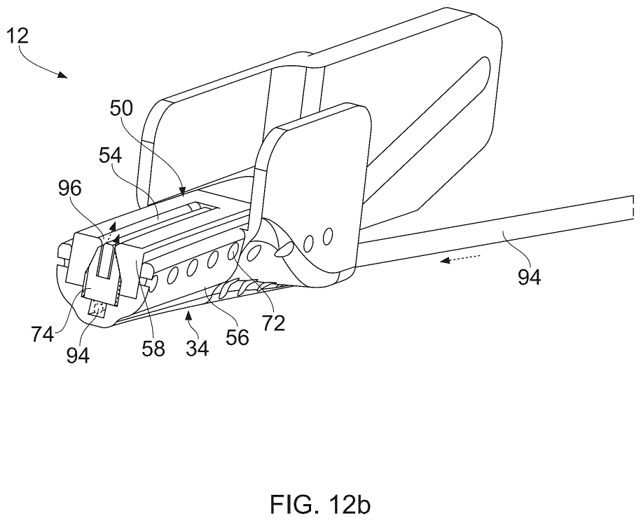

The embodiment of the electrosurgical instrument 12 shown in FIGS. 12a and 12b includes the same features, characteristics, and/or optional embodiment as the embodiment of the electrosurgical instrument 12 shown in FIG. 11 except for the following differences.

The first electrode 54 is not supported by the first isolating portion 58 but by the electrode support 74 which is attached to the second electrode 56. The electrode support 74 is made from an elastically non-deformable and electrically isolating material, such as a ceramic. Thus, the first electrode 54 is not floating, e.g. movable with respect to the second electrode 56.

The second electrode 56 includes a channel which is closed by the electrode support 74 for providing a section of the liquid feed 94. The electrode support 74 does not tightly seal the channel in the second electrode 56. Further, the gap is provided between the electrode support 74 and the first isolating portion 58. Again, the gap between the electrode support 74 and the first isolating portion 58 may be so thin that the liquid is sucked by capillary action from the liquid output 94 to the first surface. As such, the gap can provide a constant supply of liquid at the first surface 50.

1. An electrosurgical instrument for sealing or cutting tissue, comprising
an instrument shaft comprising a transmission line for conveying microwave or [and/or] radiofrequency electromagnetic energy;
a first jaw attached to the instrument shaft and including a first surface,
a second jaw attached to the instrument shaft and including a second surface,
a first electrode for emitting microwave or radiofrequency energy,
a second electrode for emitting microwave or radiofrequency energy,
a third electrode for emitting microwave or radiofrequency energy, and
a first isolating portion electrically isolating the first electrode from the second electrode,
wherein the first jaw and the second jaw can be moved between an open position, in which the tissue can be inserted between the first surface and the second surface, and a closed position, in which the first and second surfaces are brought together to clamp the tissue therebetween,
wherein the first electrode and the second electrode are arranged on the first jaw,
wherein the third electrode is arranged on the second jaw,
wherein the first isolating portion is made from an elastically deformable material,
wherein the first electrode is attached to and supported by the first isolating portion so that the first electrode is movable relative to the second electrode upon application of pressure on the first electrode, and
wherein the first electrode is made from an electrically conductive material and interlocks with the first isolating portion.

2-4. (canceled)

5. The electrosurgical instrument according to claim 1, wherein the first electrode includes at least one through-hole which is filled with the first isolating portion.

6. The electrosurgical instrument according to claim 1, wherein the first electrode includes a base plate and a rib arranged on the base plate for forming a T-shape in a cross-sectional view of the first electrode,
wherein at least a part of the base plate on each side of the rib and facing the second jaw is covered by the first isolating portion.

7. The electrosurgical instrument according to claim 6, further comprising a fifth electrode for emitting microwave and arranged on the second jaw,
wherein the fifth electrode includes a base plate and a rib arranged on the base plate for forming a T-shape in a cross-sectional view of the fifth electrode, and
wherein the first electrode is completely embedded within the first isolating portion or the fifth electrode is completely embedded within a second isolating portion arranged in a channel of the third electrode.

8. The electrosurgical instrument according to claim 7, wherein, in the closed position, the ribs of the first electrode and the fifth electrode are spaced away from each other along a direction perpendicular to a longitudinal direction.

9. The electrosurgical instrument according to claim 8, further comprising a blade movable parallel and between the first electrode and the fifth electrode in the closed position.

10. The electrosurgical instrument according to claim 6, further comprising a blade movable parallel to the first electrode in the closed position, the blade being arranged on the second jaw and protruding from the second surface,
wherein a gap is provided between the rib and the first isolating portion, and
wherein the blade is movable in the gap in the closed position.

11. The electrosurgical instrument according to claim 10, wherein the blade is electrically isolated from the third electrode and spatially offset from the first electrode in the closed position,
wherein the blade is electrically connected to the transmission line to provide a counter electrode to the first electrode for radiofrequency cutting between the first electrode and the blade.

12. The electrosurgical instrument according to claim 1, further comprising a fourth electrode for emitting microwave or radiofrequency energy and arranged on the first jaw or a fifth electrode for emitting microwave or radiofrequency energy and arranged on the second jaw,
wherein the fourth electrode or the fifth electrode each have a U-shape in a cross-sectional view.

13. The electrosurgical instrument according to claim 12, further comprising a second isolating portion arranged in a channel of the third electrode,
wherein the fifth electrode is completely embedded within the second isolating portion or the fourth electrode is completely embedded within the first isolating portion.

14. The electrosurgical instrument according to claim 12, further comprising a second isolating portion arranged in a channel of a third electrode,
wherein end faces of the fourth electrode are at least partially exposed on the first surface, exposed sections of the fourth electrode being offset to the first electrode, or
end faces of the fifth electrode are at least partially exposed on the second surface, the exposed sections of the fifth electrode being offset to the first electrode in the closed position.

15. The electrosurgical instrument according to claim 14, wherein the exposed sections of the fourth electrode are arranged between sections of the second electrode that are exposed on the first surface, or
the exposed sections of the fifth electrode are arranged between sections of the third electrode that are exposed on the second surface.

16. The electrosurgical instrument according to claim 1, further comprising a sixth electrode which extends parallel to the first electrode on the first jaw for providing RF cutting between the first electrode and the sixth electrode,
wherein optionally the second electrode is electrically connected to the third electrode and a fourth electrode is electrically connected to a fifth electrode so that microwave energy is generated between a pair of second electrode and third electrode and the pair of fourth electrode and fifth electrode.

17. The electrosurgical instrument according to claim 16, wherein the first electrode and the sixth electrode are made from an electrically conductive material or the sixth electrode is mirror symmetric to the first electrode.

18. The electrosurgical instrument according to claim 16, further comprising an electrode support made form an electrically isolating material, the electrode support including a support base plate and a support rib arranged on the support base plate for forming a T-shape in a cross-sectional view of the first jaw,
wherein the first electrode and the sixth electrode each include a strip made from an electrically conductive material and arranged on opposing sides of the support rib.

19. The electrosurgical instrument according to claim 18, wherein the support base plate is in contact with the fourth electrode.

20. The electrosurgical instrument according to claim 12, further comprising a second isolating portion arranged in a channel of the third electrode,
wherein the fifth electrode interlocks with the second isolating portion and is exposed on the second surface so that the fifth electrode contacts the first electrode in the closed position.

21. The electrosurgical instrument according to claim 1, further comprising a liquid output and a liquid feed for supplying liquid to the liquid output,
wherein the liquid output is arranged between the first electrode and the first isolating portion for wetting the first surface at the first electrode.

22. The electrosurgical instrument according to claim 21, wherein the liquid output includes a plurality of liquid outlets along the first electrode or
a single outlet extending along a section of the first electrode.

23. An electrosurgical apparatus for sealing or cutting tissue, comprising:
a generator unit for generating radiofrequency or microwave electromagnetic energy, and
the electrosurgical instrument according to claim 1,
wherein the transmission line is configured to convey the radiofrequency or microwave electromagnetic energy from the generator unit to the first electrode, the second electrode, the third electrode, a fourth electrode, a fifth electrode or a sixth electrode.
